(19) United States
(12) Patent Application Publication
(10) Pub. No.: US 2005/0171424 A1
(43) Pub. Date: Aug. 4, 2005

(54) Methods for imaging the lymphatic system using dendrimer-based contrast agents

(75) Inventors: Martin W. Brechbiel, Annandale, VA (US); Hisataka Kobayashi, Nishinomiya (JP); Peter L. Choyke, Bethesda, MD (US); John C. Morris, Rockville, MD (US); Thomas A. Waldmann, Silver Spring, MD (US)

Correspondence Address: KLARQUIST SPARKMAN, LLP, 121 S.W. SALMON STREET, SUITE #1600, ONE WORLD TRADE CENTER, PORTLAND, OR, 97204-2988, US

(73) Assignee: The Gov. of the USA as rep. by the Secretary of the Dept. of Health and Human Services

(21) Appl. No.: 10/756,948

(22) Filed: Jan. 13, 2004

(51) Int. Cl. A61B 5/05; A61B 5/055

(52) U.S. Cl. 600/420; 424/9.3

(57) ABSTRACT

Methods are disclosed for lymphatic system imaging using dendrimer conjugates as contrast agents. The disclosed methods are applicable to the imaging of all lymphatic structures, but in particular embodiments are particularly suited for imaging specific parts of the lymphatic system such as lymph nodes or lymphatic vessels. The methods permit the assessment of abnormal conditions within the lymphatic system, such as lymphoma/lymphoproliferative disease, inflammation, and cancer metastasis. The methods also may be used to identify and locate lymph nodes into which lymph fluid flows from a tumor.

### FIELD

Methods of imaging the lymphatic system are disclosed. More specifically, the disclosure relates to magnetic resonance imaging (MRI) methods for imaging the lymphatic system using dendrimer-based MRI contrast agents.

### BACKGROUND

The lymphatic system is the network of circulatory vessels or ducts in which the interstitial fluid bathing the cells of all tissues (except nerve tissue) is collected and carried to join the bloodstream proper. The lymphatic system is of importance in transporting digested fat from the intestine to the bloodstream, in removing and destroying toxic substances and in resisting the spread of disease throughout the body. Lymphatic capillaries are more permeable than ordinary blood capillaries, so molecules too large to directly enter the blood stream will pass into the lymphatic system for transport away from tissues. The lymphatic capillaries run together to form larger ducts that intertwine about the arteries and veins. The lymph in these larger ducts is moved along by the muscular movements of the body as a whole, and is prevented from flowing back through the ducts by one-way valves located at intervals along the ducts. Also along the course of the lymphatic vessels are situated the lymph nodes (LNs), also called the lymph glands. These nodes are bean-shaped organs containing large numbers of leukocytes embedded in a network of connective tissue. All the lymph being returned along the lymphatic system to the bloodstream must pass through several of these nodes, which filter out infectious and toxic material and destroy it. The nodes serve as a center for the production of phagocytes, which engulf bacteria and poisonous substances, and during the course of any infection, the nodes become enlarged because of the large number of phagocytes being produced. Since certain malignant tumors also tend to spread through the lymphatic system, surgical removal of all nodes that are suspected of being involved in the spread of such malignancies is an accepted but undesirable therapeutic procedure.

Sentinel node biopsy is a technique used to determine more accurately whether a cancer has spread (metastasized), or is localized to the primary tumor. The “sentinel” lymph node (SLN) is the first lymph node along one or more paths of lymphatic drainage away from the primary tumor before lymphatic flow drains secondarily into the remaining regional LNs. A negative biopsy and analysis of the SLN(s) for metastatic cells reliably indicates that a cancer has not metastasized, and may spare a cancer patient more drastic treatments and procedures. However, identifying and locating the SLN can be difficult, and prior to more recent methods for identifying the SLN, all of the regional LNs near a tumor were removed for analysis by a pathologist. In many patients, the regional LNs turned out to be free of tumor cells, but these patients were still placed at risk of developing the potential complications of major LN resection, including chronic swelling, discomfort, reduced mobility, and increased risk of infection. The SLN concept has been applied successfully to the treatment of a variety of cancers, including cancer of the penis, skin, breast, vulva, lung, head and neck (including papillary thyroid carcinoma).

Breast cancer is the most common malignancy in women, resulting in approximately 45,000 deaths annually in the United States (see, for example, Landis et al., “Cancer Statistics, 1999,” CA Cancer J. Clin., 49: 8-31, 1999). The presence of lymph node metastases has major negative prognostic implications in breast cancer patients, and is the major criterion for determining the need for adjuvant chemotherapy. For many years, surgical dissection of the axillary lymph nodes was used to assess lymph node involvement by breast cancer. Now, at least two accepted methods exist for identifying and locating sentinel lymph nodes associated with breast and other types of cancer.

The commonly used methods for identifying and locating the SLN employ peritumoral injections of either isosulfan blue dye, or a radionuclide-labeled sulfur or albumin colloid (radiocolloid). The dye or radiocolloid serves as a tracer of lymphatic flow away from a tumor. In the blue dye technique, the SLN is detected by direct visualization, which requires blind dissection of tissue until the “dyed” SLN is detected. In the radiocolloid technique, the SLN is located based on a localized accumulation of radioactivity that is detected using a hand-held gamma ray counter (see, for example, Alazraki et al., “Sentinel Node Staging of Early Breast Cancer Using Lymphoscintigraphy and the Intraoperative Gamma-Detecting Probe,” Update on Nuclear Medicine, 39: 947-956, 2001). The radionuclide method can assist in localization of the SLN, but it has poor spatial resolution. Therefore, the surgeon still has to search through tissue to locate the SLN.

The dye and radionuclide methods may be combined, with the radionuclide used to find the general area of the SLN and the dye used to help the surgeon locate the exact position of the SLN within that general area. Still, a LN with high radioactivity and/or intense blue staining is not necessarily a SLN since the radiocolloids and blue dye tend to move away from the actual SLN to more distant LNs during the procedures. A “first appearance criterion” has been applied to identify a SLN as a node that is first in time to receive a dye or radiocolloid that has been injected into or near a tumor. However, the dye and radiocolloid methods offer insufficient temporal resolution to assure reliable SLN identification based on this criterion.

Magnetic resonance imaging (MRI) has been proposed as a method for identifying SLNs based on a first appearance criterion, and a number of magnetic resonance contrast agents have been tried for lymphangiography (visualization of the lymph system and lymph flow therein). For example, Suga et al. used the low molecular weight contrast agent gadopentetate dimeglumine (GPDM) to image lymph flow (see, Suga et al., “Localization of Breast Sentinel Lymph Nodes by MR Lymphography with a Conventional Gadolinium Contrast Agent,” Acta Radiologica, 44: 35-42, 2003). GPDM does not, however, exhibit lymphotropic properties, and the lymphatic distribution of this compound appears to be unpredictable and inconsistent enough to preclude its use in a clinical setting.

Ultra-small iron oxide particles (USPIO) and Gadomer-17 have been used as contrast agents for lymphangiography. In the case of USPIO, it has been reported that MRI with this contrast agent does not permit observation of small lymph nodes (see, Hoffman et al., “Functional Magnetic Resonance Imaging Using Iron Oxide Particles in Characterizing Head and Neck Adenopathy” Laryngoscope, 110:1425-1430, 2000). Furthermore, since USPIO provides negative contrast of lymph nodes relative to surrounding tissue, it is not compatible with image-guided dissection or biopsy of lymph nodes. The use of Gadomer-17 contrast agent has been somewhat more successful for locating and identifying SLNs, and for enabling image-guided procedures (see, Torchia & Misselwitz, “Interstitial MR Lymphangiography for the Detection of Sentinel Lymph Nodes,” J. Surgical Oncology, 78: 151-156, 2001). Nonetheless, neither of these MRI methods provides images of lymphatic system structure with sufficient spatial resolution to permit direct non-invasive assessment of disease states in the lymphatic system (i.e. without biopsy).

### SUMMARY

Methods are disclosed for imaging and non-invasive disease state assessment of the lymphatic system. The methods employ dendrimer conjugates as MRI, CT contrast agents or lymphoscintigraphy agents, and can provide highly detailed images of lymphatic structures that permit assessment and differentiation of several disease states, including inflammation, infection, proliferative disorder, and tumor metastasis into lymph nodes. In certain embodiments, the disclosed methods also are useful for accurate localization of particular lymphatic structures during image-guided procedures, for example, during image-guided needle biopsy of a particular lymph node. Furthermore, time-series of such detailed images enable highly accurate assignment of sentinel node status based on the first appearance criterion. Comparative results demonstrate that the disclosed methods are surprisingly superior to methods of imaging the lymphatic system with GPDM and Gadomer-17.

In some embodiments, the disclosed methods include administering to a subject an image-enhancing amount of a dendrimer conjugate where the dendrimer conjugate is a conjugate of a DAB-G4D, DAB-G5D, DAB-G6D, DAB-G7D, DAB-G8D, PAMAM-G4D, PAMAM-G5D, PAMAM-G6D, PAMAM-G7D, or PAMAM-G8D dendrimer and a metal chelate. A difference in a magnetic resonance signal intensity of at least a portion of the lymphatic system is detected and differences in signal intensity may be used to detect structural and/or functional features of the lymphatic system.

### BRIEF DESCRIPTION OF THE DRAWINGS

FIG. 1 is a schematic representation of the general structures of lower generation DAB-Am and PAMAM dendrimers; higher generation DAB-Am and PAMAM dendrimers have similar structures, but are larger with additional branches and terminal amino groups. As shown, a doubling of the branches and the number of terminal amino groups occurs with each successively higher generation.

FIG. 2 is a magnetic resonance image and a schema of the imaged structures in the region of the mammary gland that was obtained 36 min after injection of a PAMAM-G6 dendrimer conjugate.

FIG. 3 is a series of 3D dynamic mammo-lymphangiograms obtained following the sequential injection of GPDM and the PAMAM-G6 contrast agent (approximately 36 minutes later; see the time course inset) showing the lack of enhancement of lymphatic structures in the absence of the PAMAM-G6 agent, and the remarkable image contrast obtained for the lymphatics draining the mammary gland following administration of the PAMAM-G6 agent.

FIG. 4A is set of 2D-fastIR stereo-view images of a BALB-neuT transgenic mouse bearing a bilateral breast tumor (solid arrows) and two metastatic tumors (broken arrows) in the axilla and the lateral chest wall that was obtained following administration of the PAMAM-G6 contrast agent. A schema of the images also is shown to aid in the interpretation of the images.

FIG. 4B is a series of 3D dynamic mammo-MR-lymphangiograms obtained for the same mouse as shown in FIG. 4A that was obtained following administration of the PAMAM-G6 contrast agent. Several dilated lymphatic vessels extending from the breast tumor to two tumors in lymph nodes at the lateral chest wall were clearly imaged.

FIG. 5A is a set of 2D-fastIR stereo-view images of a mouse with a PT-18 tumor (solid arrow) in the breast and a tumor (broken arrow) in the axillary lymph node obtained following administration of the PAMAM-G6 contrast agent.

FIG. 5B is a series of 3D mammo-MR-lymphangiograms of the same mouse shown in FIG. 5B showing that the axillary lymph node tissue with metastatic tumor cells did not show enhancement by the PAMAM-G6 contrast agent. However, the lymphatic vessel flowing into the lymph node with a metastatic tumor was dilated and showed enhancement. A schema to aid interpretation of the images is also shown as an inset.

FIG. 6A is a pair of 3D mammo-MR-lymphangiograms of axillary lymph nodes without (left image) and with (right image) a PT-18 metastatic tumor showing, with surprising detail, the lack of filling of the metastatic lymph node and dilation of the afferent lymph vessel of the lymph node.

FIG. 6B is a pair of histological sections (hematoxylin-eosin stained) confirming tumor growth in the non-enhanced portion of the metastatic lymph node (right-hand section, corresponding to the right-hand image of FIG. 6A) compared to the normal lymph node which showed no filling defects (left-hand section, corresponding to the left-hand image of FIG. 6A).

FIG. 7 shows, on the left, a typical 3D-micro-MR lymphangiogram of normal mice taken 45 minutes after administration of the PAMAM-G8 contrast agent. A schema that aids in interpretation of the MR image is shown on the right. The injection site and locations of the components of the deep lymphatic system are indicated by the labeled arrows.

FIG. 8A is a set of whole-body 3D-micro-MR MIP images of mice injected intracutaneously in all four middle phalanges with 0.005 mmolGd/kg of PAMAM-G8, DAB-G5, PAMAM-G4, Gadomer-17 or GPDM taken at 10 minutes after injection, showing the superior image detail obtained with the disclosed dendrimer contrast agents in comparison to both Gadomer-17 and GPDM.

FIG. 8B is a set of whole-body 3D-micro-MR MIP images of the same mice imaged in FIG. 8A, only at 45 minutes after injection, showing the persistent superior image detail obtained with the disclosed dendrimer contrast agents in comparison to both Gadomer-17 and GPDM.

FIG. 9A is a graph showing the axillary lymph node-to-muscle image signal intensity ratio for PAMAM-G8, DAB-G5, PAMAM-G4, Gadomer-17, and GPDM over time following administration of each of the contrast agents. The values are expressed as the mean and the standard deviation (N=5 or 6). The asterisks indicate significant differences from the group with PAMAM-G8 (P<0.01).

FIG. 9B is a graph showing the axillary lymph node-to-liver image signal intensity ratios for PAMAM-G8, DAB-G5, PAMAM-G4, Gadomer-17, and GPDM over time after administration of each of the contrast agents. The values are expressed as the mean and the standard deviation (N=5 or 6). The asterisks indicate significant differences from the group with PAMAM-G8 (P<0.01).

FIG. 10 is a pair of whole-body dynamic 3D-micro-MR lymphangiograms of a mouse with Concanacalin A lymphangitis that was injected intracutaneously into all four middle phalanges with 0.005 mmolGd/kg of PAMAM-G8, and imaged at 10 and 45 minutes following administration of the contrast agent.

FIG. 11 is a pair of whole-body 3D-micro-MR lymphangiograms of IL-15 transgenic mice (high producer) with induced lymphoadenopathy and subcutaneous involvement of lymphoproliferative disorder that were obtained 45 minutes after administration of (left) 0.005 mmolGd/kg of PAMAM-G8 and (right) DAB-G5. Dilation of subcutaneous lymphatic vessels (broken arrows) and swollen right axillary lymph nodes (solid arrows) are indicated on the images.

FIG. 12A is a whole-body 3D-micro-MR lymphangiogram of a mouse with a lymph node metastasis obtained 45 minutes after injection of 0.005 mmolGd/kg of PAMAM-G8. Large inguinal and abdominal tumors (asterisks) accompanied by the left inguinal lymph node (long arrow) are shown, along with dilated lymphatic vessels surrounding the tumor and a collateral lymphatic vessel, which communicated with the thoracic duct via the axillary lymph node (arrowheads).

FIG. 12B is a whole-body 3D-micro-MR lymphangiogram of a mouse with a subcutaneously xenografted tumor of MC-38 cells that was obtained 45 minutes after injection of 0.005 mmolGd/kg of PAMAM-G8. A large inguinal tumor (astersisk) is shown accompanied by the left normal inguinal lymph node (long arrow). No dilated lymphatic vessels surrounding the tumor are seen.

FIGS. 13A and 13B are whole-body 3D-micro-MR lymphangiograms of normal mice given intracutaneous injections into all four middle fingers with 0.05 mmolGd/kg of PAMAM-G8 and GPDM, respectively.

FIG. 14A is a pair of whole-body 3D micro-MR-lymphangiograms (stereo view) of a mouse with concanavilin-A-induced lymphangitis showing dilated lymph vessels (arrows).

FIG. 14B is a two-dimensional micro-MR image of the liver of the mouse shown in FIG. 14A having concanavalin-A lymphangitis, showing enhancement adjacent to the vascular structures (arrows), which did not show enhancement.

FIG. 14C is a histological microscope picture (20×) of the region of the mouse's liver shown in FIG. 14B showing that lymphocytes had mainly infiltrated adjacent to the vascular structures (arrows) in the same place where enhancement was shown in FIG. 14B.

FIG. 15A is a composite of a whole-body 3D-micro-MR and neck and pelvic 2D micro-MR lymphangiograms of a mouse with IL-15 transgenic-induced lymphoadenopathy with CD8⁺ T-cells taken 45 minutes after intracutaneous injection of PAMAM-G8 into the fingers of the mouse showing enlargement and a lack of enhancement within several of the lymph nodes.

FIG. 15B is a microscopic picture (20×) of an enlarged lymph node obtained from the IL-15 transgenic mouse in FIG. 15A, showing that the germinal center structure of the lymph node was no longer seen and was replaced by a homogeneous dense infiltration of lymphoid cells.

FIG. 16 is a composite of three 3D-micro MR images of the external iliac lymph nodes in normal mice (A), in nude mice that spontaneously develop oral ulcers and urinary tract infections (B), and in IL-15 transgenic mice with lymphoproliferative or neoplastic disease (C). These images were taken 45 minutes after administration of PAMAM-G8 and demonstrate that the disclosed methods can be used to differentiate infectious and neoplastic changes in the lymph nodes.

FIG. 17 is a schematic diagram showing an exemplary magnetic resonance instrument for performing the disclosed methods.

### DETAILED DESCRIPTION

In order to facilitate review of the various embodiments of the invention, the following explanations of specific abbreviations and terms are provided:

SLN—sentinel lymph node

IL-15—interleukin-15

NK—natural killer

IEL—intraepithelial lymphocyte

CT—X-ray computed tomography

MR—magnetic resonance

MRI—magnetic resonance imaging

MRL—magnetic resonance lymphangiography

dmMRML—dynamic micro-magnetic resonance mammo-lymphangiography

2D-micro-MRL—two-dimensional micro-magnetic resonance lymphangiography

3D-micro-MRL—three-dimensional micro-magnetic resonance lymphangiography

2D-fastIR—two-dimensional fast-inversion recovery

SPGR—spoiled gradient echo

MIP—maximum intensity projection

1B4M—2-(p-isothiocyanatobenzyl)-6-methyl-diethylenetriamine pentaacetic acid

USPIO—ultra-small particle of iron oxide

DAB—diaminobutane

DTPA—diethylenetriaminepentaacetic acid

GPDM—gadopentetate dimeglumine (Gd-DTPA-dimeglumine), a low molecular weight (0.94 kDa), FDA-approved extracellular MRI contrast agent also known as Magnevis™ (Schering AG, Berlin, Germany).

Gadomer-17—a low molecular weight (17 kDa) dendrimer-based magnetic resonance imaging agent (Schering AG, Berlin, Germany)

PAMAM—polyamidoamine

1B4M—2-(p-isothiocyanatobenzyl)-6-methyl-diethylenetriaminepentaacetic acid

DAB-G4D—generation-4 DAB-Am dendrimer

DAB-G5D—generation-5 DAB-Am dendrimer

DAB-G6D—generation-6 DAB-Am dendrimer

DAB-G7D—generation-7 DAB-Am dendrimer

DAB-G8D—generation-8 DAB-Am dendrimer

PAMAM-G4D—generation-4 PAMAM dendrimer

PAMAM-G5D—generation-5 PAMAM dendrimer

PAMAM-G6D—generation-6 PAMAM dendrimer

PAMAM-G7D—generation-7 PAMAM dendrimer

PAMAM-G8D—generation-8 PAMAM dendrimer

DAB-G4—DAB-Am-64-(Gd-1B4M)₆₄ dendrimer conjugate

DAB-G5—DAB-Am-128-(Gd-1B4M)₁₂₈ dendrimer conjugate

DAB-G6—DAB-Am-256-(Gd(1B4M)₂₅₆ dendrimer conjugate

DAB-G7—DAB-Am-512-(Gd-1B4M)₅₁₂ dendrimer conjugate

DAB-G8—DAB-AM-1024-(Gd-1B4M)₁₀₂₄ dendrimer conjugate

PAMAM-G4—PAMAM-G4D-(Gd-1B4M)₆₄ dendrimer conjugate

PAMAM-G5—PAMAM-G5D-(Gd-1B4M)₁₂₈ dendrimer conjugate

PAMAM-G6—PAMAM-G6D-(Gd-1B4M)₂₅₆ dendrimer conjugate

PAMAM-G7—PAMAM-G7D-(Gd-1B4M)₅₁₂ dendrimer conjugate

PAMAM-G8—PAMAM-G7D-(Gd-1B4M)₁₀₂₄ dendrimer conjugate

Unless otherwise explained, all technical and scientific terms used herein have the same meaning as commonly understood by one of ordinary skill in the art to which this invention belongs. Definitions of common terms in magnetic resonance imaging may be found, for example, in Bushong, Magnetic Resonance Imaging: Physical and Biological Principles, Mosby, 1996. In the case of conflict, terms have the meanings provided in the present disclosure.

The singular terms “a,” “an,” and “the” include plural referents unless context clearly indicates otherwise. Similarly, the word “or” is intended to include “and” unless the context clearly indicates otherwise. The term “comprises” means “includes.” Molecular weights and formulas specifically recited are for illustrative purposes, and one of ordinary skill in the art will recognize that either may vary in practice from those specifically recited.

The term “dendrimer conjugate” refers to a dendrimer attached to one or more metal chelates.

The term “dendrimer” refers to a class of highly branched, often spherical, macromolecular polymers that exhibit greater monodispersity (i.e. a smaller range of molecular weights, sizes, and shapes) than linear polymers of similar size. These three-dimensional oligomeric structures are prepared by reiterative reaction sequences starting from a core molecule (such as diaminobutane or ethylenediamine) that has multiple reactive groups. When monomer units, also having multiple reactive groups, are reacted with the core, the number of reactive groups comprising the outer bounds of the dendrimer increases. Successive layers of monomer molecules may be added to the surface of the dendrimer, with the number of branches and reactive groups on the surface increasing geometrically each time a layer is added. The number of layers of monomer molecules in a dendrimer may be referred to as the “generation” of the dendrimer. The total number of reactive functional groups on a dendrimer's outer surface ultimately depends on the number of reactive groups possessed by the core, the number of reactive groups possessed by the monomers that are used to grow the dendrimer, and the generation of the dendrimer.

The term “metal chelate” refers to a complex of a metal ion and a group of atoms that serves to bind the metal ion (a “metal chelating group”). Typically, the metal chelating groups are attached to reactive groups on the surface (located, for example, at the termini of the dendritic branches) of the dendrimer. In some embodiments, a dendrimer conjugate may have fewer bound metal ions than it has metal chelating groups on its surface. For example, in particular embodiments at least 25%, 50%, 75%, 90% or 95% of the metal chelating groups may have bound metal ions. Similarly, dendrimer conjugates may have fewer metal chelating groups than there are surface reactive groups on the dendrimer. For example, in particular embodiments at least 25%, 50%, 75%, 90% or 95% of the surface groups of a dendrimer may be bonded to a metal chelating group. The differences in the number of metal chelates and the number of bound metal ions lead to the above-mentioned differences in chemical formulae and molecular weights.

The term “PAMAM dendrimer” refers to a dendrimer having polyamidoamine branches. As used herein, the term “DAB dendrimer” refers to a dendrimer having a diaminobutane core and polyalkylenimine branches. In general, DAB dendrimers may have polyalkylenimine branches, such as polyethyleneimine, polypropylenimine and polybutyleneimine branches. The term “DAB-Am dendrimer” refers to a DAB dendrimer having polypropylenimine branches and one or more surface amino groups, that is, amino groups at the ends of the last layer of branches that are added to the dendrimer as it is grown from the initiator core are terminated with one or more reactive amine groups. For example, when a DAB-Am dendrimer is synthesized using alkylenimine monomers, each successive layer of monomers that is added to the growing dendrimer to form additional branches provides a doubling of the number of free amine groups at the ends of the branches. The free amine groups at the ends of the branches (the surface of the dendrimer) may either be used as the reactive sites for adding an additional layer of monomers to the dendrimer to increase its generation or may be derivatized to provide alternative functional groups, such as quaternary amine groups or amide groups, on the surface of the dendrimer. Dendrimers of a particular generation and internal structure (core and branch structure), but with differing functional groups on their surfaces are commercially available.

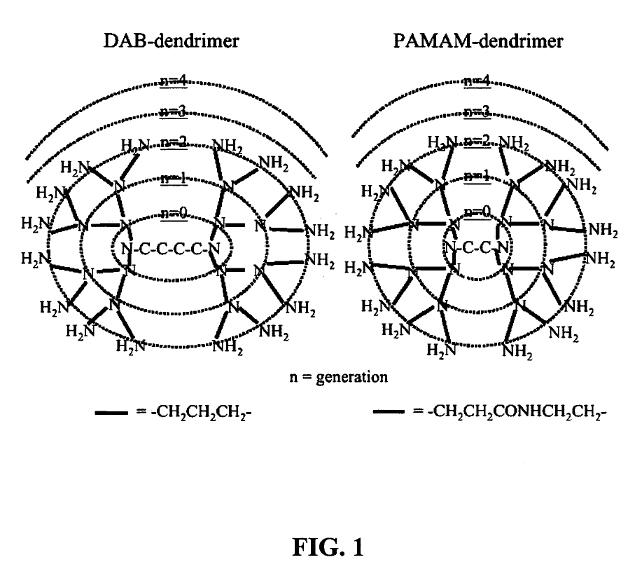

The term “DAB-Am-X” refers to a DAB-Am dendrimer having X number of surface amino groups. For example, DAB-Am-64 denotes a diaminobutane-core dendrimer having polypropylenimine branches and 64 amino groups at its surface. The structures of specific low-generation PAMAM dendrimers and low-generation DAB-Am dendrimers are compared in FIG. 1. FIG. 1 also illustrates the geometric increase in the number of branches and terminal amino groups with each successively higher generation of dendrimer. Of course, such amino groups appear as free (or surface) amino groups only at the ends of the branches. Otherwise in FIG. 1 internal amino groups are shown reacted with and bonded to additional branches that extend outward.

The term “bifunctional chelating agent” refers to a molecule that has at least two functional groups, one of which is a reactive group which can form a bond, such as a covalent bond, with another molecule, and one of which is a metal chelating group. Bifunctional chelating agents may be reacted with dendrimers to provide dendrimer conjugates, with metals added to the metal chelating group of the bifunctional chelating agent either before or after reaction of the bifinctional chelating agent with the dendrimer. Conjugation between a dendrimer and a metal chelate typically refers to formation of a covalent bond between the dendrimer and the metal chelate(s). However, in some instances ion-ion bonds, ion-dipole bonds, dipole-dipole bonds and hydrophobic interactions may be used to conjugate a dendrimer and a metal chelate.

The disclosed dendrimer conjugates are useful for imaging the lymphatic system of a subject (for example, a mammal, such as a human or veterinary animal, including a horse, a cow, a sheep, a pig or a mouse). Therefore, in one embodiment, a method for lymphatic system imaging is provided. The method includes administering an image enhancing amount of a dendrimer conjugate to a subject.

Any imaging technique, including MRI, CT, and lymphoscintigraphy may be used. However, MRI has several advantages over the other techniques for producing images of the lymphatic system. The spatial resolution of MRI (0.1-0.3 mm) is 30-100 times greater than that of scintigraphy (1 cm), and about 10 times greater than CT. Also, the temporal resolution of MRI is greater than 10 times that of scintigraphy, offering greater potential for dynamic studies of the lymphatics, for example, to identify sentinel lymph nodes based on a first appearance criterion. Moreover, three-dimensional images provided by MRI improve anatomical localization of imaged structures, and MRI does not involve exposure to radiation.

As used herein, the terms “administer” or “administering” refer to the addition of a substance to the body of a subject. For example, the disclosed dendrimer conjugates may be administered by any appropriate route, including, but not limited to, intravenous injection, parenteral injection, intracutaneous injection, intratumoral injection, peritumoral injection, injection into the lymphatic system, injection into a surgical field and subdermal injection.

The site of intravenous, parenteral or subdermal injection is desirably, but not necessarily, in close proximity (such as less than 15 cm, 10 cm or 5 cm away from) to the lymphatic system components for which images are desired.

An “image enhancing amount” refers to an amount that is sufficient to produce detectable (visually or electronically, such as by densitometry) differences in the image of lymphatic system components (such as lymph nodes and lymphatic vessels) relative to surrounding tissue at some time following administration of the dendrimer conjugate. For MRI, such differences may be detected in either a T₁- or T₂-weighted image taken at some time after the imaging agent is administered. The differences may be due to either an increase or a decrease in the intensity of the lymphatic system or a portion thereof, relative to surrounding tissue in comparison to an image obtained before administration of the agent. For example, the image intensity of one or more components of the lymphatic system will be increased (or decreased) in intensity relative to surrounding tissue by greater than about 20%, 50%, 75% or 90% when compared to an image obtained without administering or to regions of an image that are not enhanced by the contrast agent. Other anatomical structures may or may not exhibit enhancement following administration of the dendrimer conjugate.

Differences in signal intensity between the lymphatic system, parts of the lymphatic system and the surrounding tissue may be used to detect and/or differentiate one or more conditions of the lymphatic system, such as the location of particular components of the lymphatic system (including lymphatic vessels and lymph nodes), the presence of metastatic cells in lymph nodes, swelling of lymph nodes, and dilation of lymphatic vessels. In general, components of the lymphatic system will have a positive contrast (increase in image intensity) in a T₁-weigthed image relative to surrounding tissue, especially where the dendrimer conjugate is a T₁ agent. For example, where a T₁-weighted image is obtained following administration of an image enhancing amount of a disclosed dendrimer conjugate that includes gadolinium ions (which increase the longitudinal relaxation rate 1/T₁ more than the transverse relaxation rate 1/T₂), lymphatic system components will appear brighter in a T₁-weighted image than surrounding tissue. The increase in image intensity of the lymphatic system relative to surrounding tissue permits localization of the lymphatic system components in such an image. Furthermore, where a lymph node contains metastatic tumor cells, the afferent lymphatic vessel leading to the metastatic lymph node may not only brighter appear that surrounding tissue, but also larger (dilated) than afferent lymphatic vessels leading into normal lymph nodes. In addition, swollen lymph nodes that contain metastatic tumor cells may be observed to have a bright fringe and a dark center, indicative of infiltration of the metastatic tumor cells that block entrance of the dendrimer conjugates into the germinal center of the lymph node. In contrast, swollen lymph nodes caused by infection do not exhibit a lack of contrast in the center. In addition, bright images of lymphatic vessels associated with infected and swollen lymph nodes (larger by comparison to non-infected lymph nodes seen elsewhere in a particular subject or larger by comparison to lymph nodes typically seen in normal patients) may also appear irregular, and aid in identifying swelling associated with infection rather than the presence of metastatic cancer cells. Thus, differences in the image intensities associated with the different parts of an enhanced image of a lymphatic structure (relative to surrounding tissue) can be used to identify and/or differentiate conditions of the lymphatic system.

Conversely to a T₁-image, lymphatic system components will generally have negative contrast (appear darker) in a T₂-image relative to surrounding tissue. For example, where the dendrimer conjugate includes iron ions (a T₂-agent), dark lymphatic vessels and lymph nodes will appear against a bright background of surrounding tissue. Metastatic lymph nodes will appear with a dark fringe and a bright center in a T₂-weighted image, and the dark areas indicative of afferent vessels may appear dilated. Swollen lymph nodes and dilated lymphatic vessels (such as induced by infection) will appear as larger dark areas when compared to typical corresponding normal lymph nodes and non-dilated vessels.

In particular embodiments, administering an imaging enhancing amount of the dendrimer conjugate includes administering a dose between about 0.0001 mmol metal/kg of the subject's body weight and about 1.0 mmol metal/kg of the subject's body weight, for example, between about 0.001 mmol metal/kg and about 1.0 mmol metal/kg, such as between about 0.01 mmol metal/kg and about 1.0 mmol metal/kg. In other particular embodiments, image enhancing amounts of the dendrimer conjugates are provided by administering the dendrimer conjugates in dosages that are 1/50^{th} to ⅓ of the molar dosages on a dendrimer basis or 1/2500 to 1/500 of the molar dosage on a metal ion basis (such as gadolinium ion basis) as required for simple chelates such as Gd-DOTA and Gd-DPTA (which are typically administered in a range of 0.1 to 1.0 mmol Gd/kg). In other particular embodiments, a detectable difference in lymphatic system MRI image intensity may be provided by administering between about 0.0001 mmol Gd/kg and about 1.0 mmol Gd/kg, for example, administering between about 0.01 mmol Gd/kg and about 1 mmol Gd/kg such as administering between about 0.1 mmol Gd/kg and about 1 mmol Gd/kg. intravenously, parenterally, intratumorally, peritumorally, subdermally or into a surgical field.

Imaging may begin immediately or anywhere from about 1 min to about 2 hrs after administration, such as between about 3 minutes and 60 minutes after administration. Imaging, once begun, may be continued for any subsequent amount of time that facilitates analysis of the images for a particular purpose (for example, to follow flow of the lymph fluid). For example, if identification of a sentinel lymph node is desired, a single image that is obtained anywhere between 2 and 60 minutes following administration following intratumoral administration may be sufficient. On the other hand, a series of images obtained at various points in time from administration to several hours after administration may be obtained if lymphatic flow beyond the sentinel lymph node is to be imaged or if intraoperative (during a surgical procedure) localization of one or more particular lymph nodes is desired as an aid to a surgeon performing a partial or full lymphadenectomy. For example, a series of images may be obtained successively over a period of time where each image is separated by any amount of time from the instrumental limit for successive image acquisitions to minutes or hours apart, such as 5, 10, 15, 30 minutes apart or 1, 2 or 3 hours apart. Imaging may be done before or during surgery, and continued for any period during surgery, for example, to help a surgeon guide a needle to a lymph node for a biopsy. Since surgical instruments will appear brighter than surrounding tissue in a T₁-weighted, it is desirable to use a series of T₁-wieghted images in conjunction with administration of a T₁-agent, such as a dendrimer conjugate including gadolinium ions, to permit simultaneous visualization and localization of the surgical instrument and the lymphatic system component(s) on which the surgeon will act with the instrument.

In particular embodiments, the dendrimer conjugate comprises a DAB-G4D, DAB-G5D, DAB-G6D, DAB-G7D, DAB-G8D, PAMAM-G4D, PAMAM-G5D, PAMAM-G6D, PAMAM-G7D, or PAMAM-G8D dendrimer and a metal chelate. Regardless of the dendrimer conjugate used, a difference in an image signal intensity of at least a portion of the lymphatic system that appears after the dendrimer conjugate is administered is used to image the components of the lymphatic system, including lymphatic vessels and lymph nodes.

In more particular embodiments, the dendrimer of the dendrimer conjugate is DAB-G5D, PAMAM-G4D, PAMAM-G6D or PAMAM-G8D, and in more particular embodiments the dendrimer is PAMAM-G6D. These dendrimers and the ones mentioned before may be conjugated to a variety of metal chelates including DTPA metal chelates, DOTA metal chelates, DO3A metal chelates, DOXA metal chelates, NOTA metal chelates, TETA metal chelates, DOTA-N(2-aminoethyl)amide metal chelates, DOTA-N-(2-aminophenethyl)amide metal chelates, BOPTA metal chelates, HP-DO3A metal chelates, DO3MA metal chelates, or 1B4M metal chelates. The metal ion of the metal chelate may be a metal ion of a metal selected from the metals having atomic numbers of 22-29, 42, 44 and 58-70 and combinations thereof. In even more particular embodiments, where the image signal intensity is a magnetic resonance signal intensity, the metal ion of the metal chelate is chromium (III), manganese (II), iron (II), iron (III), cobalt (II), nickel (II), copper (II), praseodymium (II), neodymium (III), samarium (III), gadolinium (III), terbium (III), dysprosium (III), holmium (III), erbium (III) or ytterbium (III). In specific embodiments, the metal ion is gadolinium (III).

In other more particular embodiments, the metal chelate of the dendrimer conjugate is a 1B4M metal chelate of gadolinium (III) ions and the dendrimer conjugate is DAB-G4, DAB-G5, DAB-G6, DAB-G7, DAB-G8, PAMAM-G4, PAMAM-G5, PAMAM-G6, PAMAM-G7 or PAMAM-G8. More particularly, the dendrimer conjugate may be DAB-G5, PAMAM-G4, PAMAM-G6, or PAMAM-G8. In specific embodiments, the dendrimer conjugate is PAMAM-G6.

Any of the dendrimer conjugates that are disclosed may further comprise an optical or fluorescent moiety to aid in location of lymphatic system components during a surgical procedure. As used herein the terms “optical moiety” and “fluorescent moiety” refer to a moiety that may be visualized by the naked eye or a photon detector (for example, a charge-coupled device) by virtue of its absorption or emission of visible light, respectively. Examples of optical and fluorescent moieties include, respectively, an isosulfan blue dye or a fluorescein molecule. In the case of a fluorescent moiety, visualization of the moiety may include illumination of the moiety with ultraviolet light to stimulate emission of fluorescent photons.

Specific components of the lymphatic system that may be imaged include lymph nodes and lymphatic vessels, regardless of their location in the subject's body. In particular embodiments, a DAB-G5 or PAMAM-G4 dendrimer conjugate is used to image lymph nodes, or a PAMAM-G8 is used to image lymphatic vessels.

Also disclosed is a method for identifying a lymph node into which lymph fluid flows from a tumor, such as a breast tumor. This particular method includes administering an image-enhancing amount of a dendrimer conjugate to an intratumoral or peritumoral site of administration. A path of lymph fluid flow from the site of administration is imaged using magnetic resonance imaging to provide an image of the lymphatic system surrounding the tumor. From this image, the lymph node may be identified along the path of lymph fluid flow from the site of administration. The method also may include detecting metastatic tumor cells in the node by detecting an image filling defect of at least a portion of the sentinel node. Alternatively, the path of lymphatic flow is imaged over time (such as for periods as described above and below) to observe a lymph node that is first in time to receive the dendrimer conjugate following administration of the dendrimer conjugate to the site of administration near or in the tumor. In some embodiments, the dendrimer conjugate used for this method is DAB-G4, DAB-G5, DAB-G6, DAB-G7, DAB-G8, PAMAM-G4, PAMAM-G5, PAMAM-G6, PAMAM-G7 or PAMAM-G8. In particular embodiments, the dendrimer conjugate is DAB-G5, PAMAM-G4, or PAMAM-G6, and in specific embodiments, the dendrimer conjugate is PAMAM-G6. As before, the dendrimer conjugate may also include an optical or fluorescent moiety.

In other alternative methods, the image of an identified lymphatic structure, such as a sentinel node, may be used in an image-guided surgical method, for example, to guide a needle to the location of a sentinel node for the purpose of obtaining a portion of the sentinel lymph node for analysis for the presence of metastatic cancer cells. Optical or fluorescent moieties conjugated to the dendrimer conjugate may further assist localization of lymphatic system components during a surgical procedure, for example, intraoperatively during a procedure to remove lymphatic system components. In one embodiment, lymph nodes that are biopsied and determined to contain metastatic cancer cells may be located for removal based on their location in an image obtained according to the disclosed methods (in an image-guided technique). Fluorescent or colored moieties conjugated to the dendrimer conjugates may further assist a surgeon in locating a lymphatic system component (such as a node) during surgical removal (for example, intraoperatively).

All publications, patent applications, patents, and other references mentioned herein are incorporated by reference in their entirety. In case of conflict, the present specification, including explanations of terms, will control. In addition, the materials, methods, and examples are illustrative only and not intended to be limiting. Various embodiments are specifically illustrated by the following examples.

### EXAMPLE 1

### Preparation and Administration of a Dendrimer Conjugate to Detect and Localize a Lymph Node

This example describes MRI imaging of the lymphatic system of mice by using a PAMAM-G6D dendrimer conjugate, specifically PAMAM-G6, which is a Gd-1B4M conjugate. Imaging of lymphatic drainage associated with breast tumors using PAMAM-G6 is shown to provide sufficient temporal and spatial resolution to accurately identify and locate lymph nodes. Sentinel lymph nodes may also be identified based on a first appearance criterion using time series of images. Image-based assessment of the disease state of the components of the lymphatic system is also demonstrated.

### I. Preparation of the Contrast Agent

The generation-6 polyamidoamine (PAMAM-G6) dendrimer (Dendritech, Inc., Midland, Mich.) has an ethylenediamine core, 256 terminal reactive amino groups, and a molecular weight of 58,048 Da. The PAMAM-G6D dendrimer was concentrated to about 5 mg/ml and diafiltrated against 0.1 M phosphate buffer at pH 9. The PAMAM-G6D dendrimer was reacted with a 256-fold molar excess of 2-(p-isothiocyanatobenzyl)-6-methyl-diethylenetriamine-pentaacetic acid (1B4M) at 40° C., and maintained at pH 9 with 1 M NaOH for 24 hours. An additional equal amount of the 1B4M was added after 24 hours as a solid. The resulting preparations were purified by diafiltration using a Centricon 30 (Amicon Co., Beverly, Mass.). This resulted in over 98% of the amine groups on the dendrimer reacting with the 1B4M as determined by ¹⁵³Gd (NEN DuPont, Boston, Mass.) labeling of aliquots, as described by Kobayashi, et al. (Kobayashi et. al, “3D-Micro-MR Angiography of Mice Using Macromolecular MR Contrast Agents With Polyamidoamine Dendrimer Core With References to Their Pharmacokinetic Properties,” Mag. Res. Med., 45: 454-60 2001).

Subsequently, PAMAM-G6 dendrimer-1B4M conjugate (about 3 mg containing 4 μmol 1B4M) was mixed with 8 μmol of non-radioactive Gd(III) citrate in 0.3 M citrate buffer overnight at 40° C. The excess Gd(III) in the preparation was removed by diafiltration using a Centricon 30 filter (Amicon Co.) while simultaneously changing the buffer to 0.05 M PBS. The purified sample was diluted to 0.2 mL with 0.05 M PBS and about 5 μL was used in each mouse breast tissue. A replacement assay using ¹⁵³Gd was used to determine that 84% of the 1B4M on the PAMAM-G6 dendrimer-1B4M conjugate was indeed chelating Gd(III) atoms, as described by Kobayashi, et al., which reference is cited in the preceding paragraph.

GPDM (Magnevist™ with a molecular weight of 938 Da), an FDA-approved extracellular MRI contrast agent (Schering AG, Berlin, Germany), was used as a control.

### II. Preparation of the Mice

Normal and breast-tumor bearing mice were prepared. Twelve week-old Balb/c mice (n=5) or athymic nu/nu mice (n=9) (NCI, Frederick, Md.) were used as the normal mice.

The breast-tumor bearing mice were thirty one week-old BALB-neuT mice (n=3) transgenic for the rat HER-2/neu (Erb B2) oncogene under the control of the mouse mammary tumor virus promotor (MMTV). These mice exhibit tissue-specific expression of HER-2/neu. BALB-neuT mice were used because of their spontaneous development of bilateral breast cancers and lymph node metastases. Heterozygous female BALB-neuT mice (BALB/c background) develop mammary gland lobule hyperplasia at 5-6 weeks of age that progresses to atypical hyperplasia by 8-9 weeks, followed by in situ carcinoma by 14 weeks, becoming invasive carcinoma usually by 21 week of age (Rovero et al., “Vaccination against rat her-2/Neu p185 more effectively inhibits carcinogenesis than transplantable carcinomas in transgenic BALB/c mice,” J Immunol., 165:5133-42, 2000). Most metastatic lymph nodes from the mouse mammary pad localize in the neck, lateral thoracic, or axillary region.

Tumor xenografts of PT-18, a murine mast cell line, were introduced into the left mammary pad in athymic nu/nu mice. When 10⁷ PT-18 cells were injected in the left mammary pad of 10 athymic nu/nu mice, six mice developed tumor masses in the left axillary lymph nodes within three weeks.

### III. Adminstration of the Contrast Agent and Imaging

Mice were anesthetized with 1.15 mg sodium pentobarbital (Dainabot, Osaka, Japan), and then injected with 0.15-0.24 μmol Gd/5-8 μL of the PAMAM-G6 contrast agent into normal mammary glands or mammary tissue surrounding a tumor (peritumorally). Dynamic micro-MR images were obtained using a 1.5-Tesla superconductive magnet unit (Signa LX, General Electric Medical System, Milwaukee, Wis.) with a birdcage type coil of 3 cm diameter fixed by a custom made coil holder. [Single and double “breast coils” used for imaging breasts in humans are commercially available, for example, from GE Medical Systems (Milwaukee, Wis.) and are routinely available in outpatient MRI centers] The mice were wrapped with gauze to stabilize their body temperature and were placed at the center of the coils.

FDA-approved MR contrast agents like GPDM rarely cause serious toxicity after intravenous or subcutaneous injection. However, since adverse reactions are typically related to dose, the PAMAM-G6 contrast agent was employed at a dose that was 1/2500 of that of GPDM on a molar basis to minimize potential toxicity. Furthermore, the PAMAM-G6 agent was administered directly into the mammary gland tissue because local injection is generally safer than intravascular injection.

In order to evaluate the lymphatic drainage from the normal mammary gland of either Balb/c mice (n=5) or athymic nu/nu mice (n=5), 3D-fast spoiled gradient echo [3D-fastSPGR (efgre3d package; Signa Horizon, GE); repetition time/echo time 19.2/7.2 msec; inversion time 47 msec; 31.2 kHz, flip angle 30°, 4 excitations; 36 slice encoding steps; scan time 4 min 49 seconds] with chemical shift fat-suppression was used 6, 12, 18, 24, 30, 36, 42, and 48 min after injection of the contrast agent. The coronal images were reconstructed with 0.6-mm section thickness every 0.3-mm. The FOV was 8×4 cm and the size of the matrix was 512×256. The slice data were processed into 3D images using the maximum intensity projection (MIP) method with the same window and level (window 3500 and level 2100) (Advantage Windows, General Electric Medical System). After imaging, the mice were sacrificed by carbon dioxide inhalation.

Athymic nu/nu mice (n=4) were anesthetized and injected with 0.15 μmol Gd of GPDM into the mammary gland, and images were taken 12, 24, and 36 min after injection. Following these three images, these mice were subsequently injected with 0.15 μmol Gd of PAMAM-G6 contrast agent in the mammary gland, and images were taken at 12 and 24 min post-injection of the PAMAM-G6 contrast agent (at 48 and 60 min post-injection of GPDM).

In order to evaluate lymphatic drainage from six tumor-bearing mammary glands of three BALB-neuT transgenic mice or the PT-18 tumor-bearing mammary gland of athymic nu/nu mice (n=6), 2D fast-inversion recovery [2D-fastIR; repetition time/echo time 8000/96 msec; inversion time 150 msec; 31.2 kHz, 2 excitations; 16 slices; scan time 2 min 16 seconds] was employed to evaluate the tumor localization before injection of PAMAM-G6 contrast agent. The coronal images were reconstructed with 1.5 mm section thickness without a gap. The FOV was 8×4 cm and the size of matrix was 512×256. The slice data were processed into 3D images using the MIP method with the same window and level (window 3500 and level 2100) (Advantage Windows, GE). Then images were obtained with the 3D-fastSPGR sequence as described above.

### IV. Results

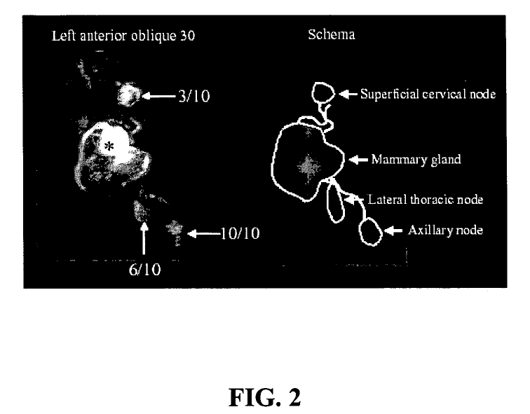

Three lymph nodes (axillary, lateral thoracic, and superficial cervical) with their draining lymphatic vessels were visualized by MRI with the PAMAM-G6 contrast agent (FIG. 2). The axillary lymph node and its afferent lymphatic vessels were visualized at the initial (6 min) time point in all 10 mice (Table 1). However, two other lymph node groups and their lymphatic vessels showed up later (Table 1). Thus, this method permitted imaging of the lymphatic system (nodes and vessels) draining normal breast tissue, and also to detect lymphatic flow over time into the cervical and lateral thoracic nodes.

TABLE 1
Visualization of Three Major Draining Lymph Nodes (LNs) From the
Normal Breast Tissue (Visualized LNs/Total Examined LNs)

| | Time (min) | | | | | | | |
|---|---|---|---|---|---|---|---|---|
| | 6 | 12 | 18 | 24 | 30 | 36 | 42 | 48 |
| Superficial | 1/10 | 1/10 | 1/10 | 2/10 | 2/10 | 3/10 | 3/10 | 3/10 |
| cervical LN | | | | | | | | |
| Lateral | 0/10 | 1/10 | 2/10 | 4/10 | 4/10 | 5/10 | 6/10 | 6/10 |
| thoracic LN | | | | | | | | |
| Axillary | 10/10 | 10/10 | 10/10 | 10/10 | 10/10 | 10/10 | 10/10 | 10/10 |
| LN | | | | | | | | |

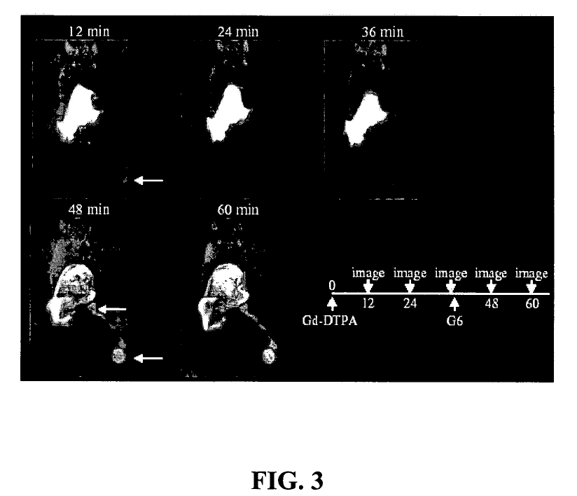

MRI images of the mice after injection of GPDM and after injection of the PAMAM-G6 contrast agent are compared in FIG. 3. Draining lymph nodes and lymphatic vessels were not well visualized at 12 minutes, 24 minutes and 36 minutes following GPDM administration. The nodes and vessels, however, were clearly visualized only after administration of the PAMAM-G6 agent 36 minutes following administration of GPDM. This result demonstrates the surprisingly superior quality of images obtained with the PAMAM-G6 contrast agent compared to GPDM for visualizing the lymphatic system with MRI.

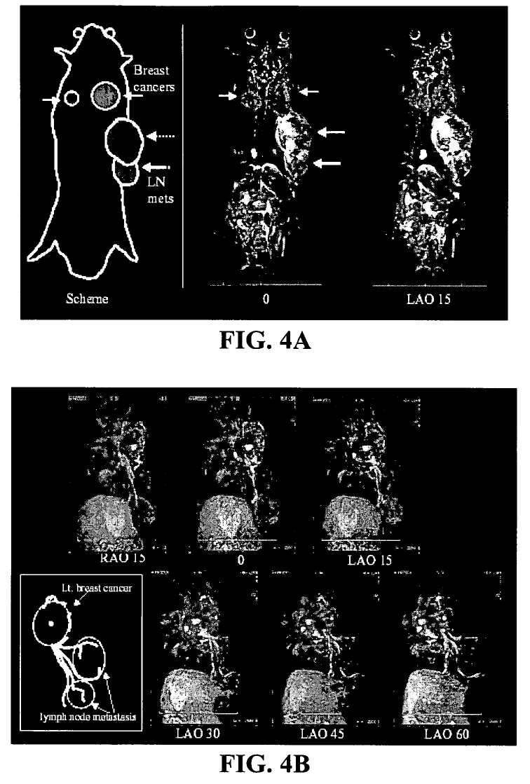

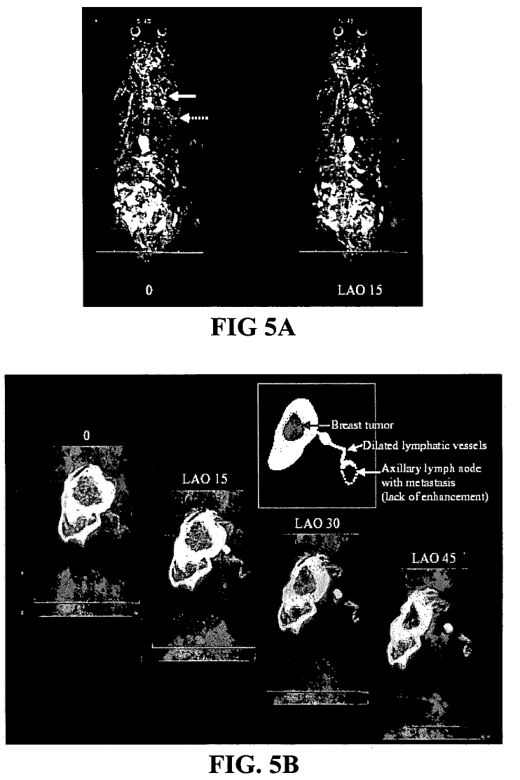

The MRI method using the PAMAM-G6 contrast agent was applied to two mouse models for breast tumors. A “spontaneous” breast cancer model using BALB-neuT transgenic mice and a PT-18 mast cell tumor xenograft injected into the breast were employed, and images of each model were obtained to visualize lymphatic drainage structure and dynamics associated with breast tumors. In both models, the flow within the draining lymphatic vessels was readily visualized (FIGS. 4 and 5).

Enlarged lymph nodes containing tumor metastases and several dilated lymphatic vessels extending from the breast tumor to two tumors in lymph nodes at the lateral chest wall were clearly imaged in the BAL-neuT model (FIG. 4). FIG. 4A shows bilateral solid tumors located in the mammary glands of a mouse and associated large, metastatic lymph nodes. FIG. 4 B is a series of 3D images that show several dilated lymphatic vessels extending from a breast tumor to lymph nodes in the lateral chest wall. These images demonstrate the precise localization of lymphatic structures afforded by the disclosed methods.

In the PT-18 model, the axillary lymph node tissue with metastatic tumor cells did not shown enhancement by the PAMAM-G6 contrast agent, but the lymphatic vessel flowing into the lymph node with a metastatic tumor was dilated and showed enhancement (FIG. 5). FIG. 5A shows a set of 2D stereo-view images of a mouse with a PT-18 tumor (solid arrow) in the breast and a tumor in the associated axillary lymph node (broken arrow). In FIG. 5B, a series of 3D images focusing (smaller field-of-view) on the breast tumor and the axillary lymph node in the PT-18 model. These images demonstrate enhancement (increase in signal intensity) of several dilated lymphatic vessels and a lack of enhancement of the interior (no increase in signal intensity) of the metastatic lymph node.

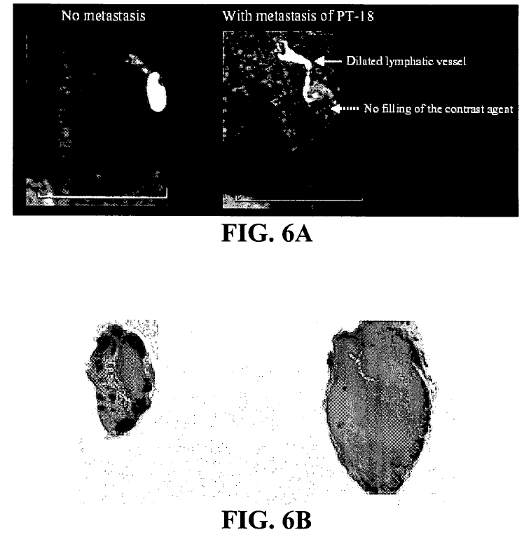

FIG. 6A shows in greater detail the differences in images (obtained the PAMAM-G6 agent) of normal lymphatics and metastatic lymphatics in the PT-18 model. The normal lymph node is much brighter and more uniformly enhanced by the dendrimer conjugate, whereas the metastatic lymph node shows a characteristic lack of enhancement in its interior. The normal afferent lymphatic vessel of the imaged lymph node is much thinner by comparison to the dilated afferent lymphatic vessel associated with the metastatic lymph node. Dilation of the lymphatic vessel in the metastatic model is believed to be due to a blockage of lymph fluid flow through the metastatic lymph node. Histopathological examination results for the normal and metastatic lymph nodes are compared in FIG. 6B, which confirm tumor growth in the non-enhanced portions of the lymph node from the PT-18 model. All 6 mice with PT-18 tumors that were studied showed abnormalities only in the axillary nodes. The axillary node was also the predominant draining node in the tumor-bearing BALB-neuT transgenic mice. Taken together, these results are consistent with the conclusion that lymphatic flow from the mouse breast drains primarily to the axillary lymph nodes.

### V. Conclusions

Sentinel lymph node localization has become a routine part of cancer surgery. Lymphoscintigraphy and intraoperative gamma probes are playing increasing roles in the surgical treatment of patients with breast cancer or malignant melanoma. However, as demonstrated in this example, MRI has potential advantages over lymphoscintigraphy. The spatial resolution of MRI (0.1-0.3 mm) is 30-100 times greater than that of scintigraphy (1 cm), and because breast MRI utilizes surface coils that substantially decrease the field of view the temporal resolution of MRI is greater than 10 times that of scintigraphy, offering a great potential for dynamic studies of the lymphatics. Furthermore, three-dimensional images improve anatomical localization; and the absence of radiation exposure is beneficial to both surgeons and patients. Therefore, dynamic mammo-MR lymphangiography can circumvent limitations of standard lymphoscintigraphy and can help distinguish the sentinel lymph node from secondary lymph nodes.

The PAMAM-G6 contrast agent is retained by or has an affinity for the normal lymph node tissue, resulting in an enhanced signal in normal lymph nodes, and the lack of enhancement in lymph nodes is a reliable sign for the presence of metastases. While the method might miss a lymph node entirely filled with tumor, most tumor bearing lymph nodes will have small rim (fringe) of normal tissue, which will be visualized as shown in FIG. 6.

FDA-approved MR contrast agents like GPDM rarely cause serious toxicity after intravenous or subcutaneous injection. Since adverse events are related to dose, the PAMAM-G6 contrast agent was employed in this Example at a dose that was 1/2500 of that of GPDM on a molar basis to further minimize potential toxicity. Furthermore, the G6 agent was administered directly into the mammary gland tissue because local injection is generally safer than intravascular injection. In order to enhance its use for potential intraoperative localization, the PAMAM-G6 agent (and any of the other disclosed dendrimer conjugates) is dual-labeled with gadolinium and an optical or fluorescent agent to help the surgeon to quickly and reliably localize the sentinel lymph node during surgery. Optical and fluorescent agents having reactive groups that permit easy conjugation of a colored or fluorescent dye to reactive groups on a dendrimer, such as surface amino, alcohol and carboxyl groups, are commercially available from Molecular Probes, Eugene, Oreg. Examples of amine reactive groups include isothiocyanates, succinimidyl esters, carboxylic acids and sulfonyl chlorides. Exemplary methods for conjugating dyes to the reactive groups on the disclosed dendrimers are provided in Haughland, Molecular Probes, Inc. Handbook of Fluorescent Probes and Research Chemicals, 9^{th} ed., 2002.

In a particular embodiment, a near-IR fluorescent dye such as Cy5.5 is conjugated to the disclosed dendrimers for the purpose of optical imaging, for example, intraoperative optical imaging to help a surgeon delineate the margins of lymphatic structures. In a more particular embodiment, a G6 dendrimer that is only partially saturated with 1B4M chelating groups, leaving a number of amine groups dispersed across the surface is prepared. A Cy5.5 dye N-hydroxysuccinimidyl active ester (Amersham Biosciences, San Francisco, Calif.) is then reacted with the remaining amine groups to provide a dendrimer conjugate that can be used for optical imaging. Optical imaging using near-IR fluorescent dyes is described, for example, in Kircher et al., Cancer Res., 63: 8122-5, 2003. Several optical imaging modalities including fluorescence reflectance imaging (FRI) and 3D quantitative fluorescence-mediated tomography are described in Bremer et al., Eur. Radiol., 13: 231-43, 2002, and an optical imaging system is described in Mahmood et al., Radiology, 213: 866-70, 1999.

The disclosed MR methods using the disclosed dendrimer-based NRI contrast agents are useful in clinical practice. The particular method using the PAMAM-G6 contrast agent that was described in this Example was able to visualize both draining lymph nodes and lymphatic vessels from breast tissue in mice. This four-dimensional imaging method helped visualize the lymphatic flow over time on a 3-D display. The superior temporal and spatial resolution of this method permits wide application of the disclosed methods to the study of tumor lymphatics and lymphatic metastasis in both experimental animals and clinical medicine.

### EXAMPLE 2

### Detection of Lymphangitis and other Lymphatic Disorders

This example compares a variety of contrast agents for MRI imaging of the deep lymphatic system and various particular components of the lymphatic system in models for a variety of lymphatic disease states.

### I. Preparation of the Contrast Agent

PAMAM-G8D, DAB-G5D, and PAMAM-G4D dendrimers (Sigma-Aldrich, St. Louis, Mo.) were each concentrated to about 5 mg/ml and diafiltrated against 0.1 M phosphate buffer at pH 9. The dendrimers were individually reacted with a 1024-, 64-, and 64-fold molar excess of 2-(p-isothiocyanatobenzyl)-6-methyl-diethylenetriamine-pentaacetic acid (1B4M), respectively, at 40° C., and maintained at pH 9 with 1 M NaOH for 24 hours. An additional equal amount of the 1B4M was added to each sample after 24 hours as a solid. The resulting preparations were purified by diafiltration using a Centricon 30 (Amicon Co., Beverly, Mass.). This resulted in over 98% of the amine groups on the dendrimers reacting with the 1B4M as determined by ¹⁵³Gd (NEN DuPont, Boston, Mass.) labeling of aliquots, as described in Example 1.

Subsequently, about 3 mg of each dendrimer-1B4M conjugate (containing 4 μmol 1B4M) was mixed with 8 μmol of non-radioactive Gd(III) citrate in 0.3 M citrate buffer, pH 4.5, overnight at 40° C. The excess Gd(III) in each preparation was removed by diafiltration using a Centricon 30 filter (Amicon Co.) while simultaneously changing the buffer to 0.05 M PBS. The purified samples were diluted to 0.2 mL with 0.05 M PBS and about 5 μL was used in each mouse extremity. A replacement assay using ¹⁵³Gd was used to determine that 80%, 85%, and 84% of the 1B4M on the PAMAM-G8, DAB-G5, and PAMAM-G4 dendrimer-1B4M conjugates, respectively, was indeed chelating Gd(III) ions (see Example 1). In brief, approximately 300,000 cpm of ¹⁵³Gd were added with 0.1 mmol of nonradioactive Gd(III) to 5 mL of the injected samples. The samples were then incubated in 0.5 M citrate buffer for 2 hours at 40° C., after which the bound and unbound fractions were separated using a PD-10 column (Pfizer, Providence R.I.).

Commercially available Gadomer-17 and GPDM (Schering AG, Berlin, Germany) were obtained and compared to the disclosed dendrimer conjugates.

### II. Preparation of the Mice

To generate a lymphangitis model, Concanavalin A (300 mg) (Sigma, St. Louis, Mo.) was injected intravenously into C57BL6 mice (NCI, Frederick, Md.) 24 hours before MRI was performed. Histological analysis of this mouse model demonstrated massive lymphocytic infiltration of most of the organs and tissues, especially in the liver, where cavernous dilation of lymphatic vessels was observed.

Eight- to 10-month old IL-15 transgenic mice (on a C57BL6 background) were used as a chronic lymphoproliferative/neoplastic disease model because they manifested selective expansion of NK, CD8⁺NK-T, γδIELs, and CD8 T cells in the periphery. Most of the lymph nodes collected from an aged IL-15 transgenic mouse were enlarged in size. The lesion of proliferated monoclonal lymphocytes often involved the lung and the subcutaneous lymphatic tissue. Pathological analysis of a lymph node from the IL-15 transgenic mouse revealed massive lymphocytic infiltration, as demonstrated by hematoylin and eosin staining. Interestingly, a typical germinal center structure was no longer seen within the lymph nodes.

In addition, 10-week-old C57BL6 mice (NCI, Frederick, Md.) bearing lymph node metastases of MC38 colorectal cancer cells following either intravenous or subcutaneous injection were used as a mouse model manifesting lymph node metastasis.

### III. Injection and Imaging

The mice were anesthetized with 1.15 mg sodium pentobarbital (Dainabot, Osaka, Japan) i.p., and then injected intracutaneously with 0.1 μmolGd of PAMAM-G8, DAB-G5, PAMAM-G4, Gadomer-17, or GPDM into four middle phalanges in all four extremities. Dynamic micro-MR images were obtained using a 1.5-Tesla superconductive magnet unit (Signa LX, General Electric Medical System, Milwaukee, Wis.) with a round birdcage type coil of 3 cm diameter fixed by a custom-made coil holder. Four to eight female mice (7 weeks old, 18-21 g body weight) in each group were used and each contrast agent was prepared at least three separate times for these imaging studies. The mice were wrapped with gauze to stabilize their body temperature and were placed at the center of the coils.

A 3D-fast spoiled gradient echo [3D-fastSPGR (efgre3d package; Signa Horizon, GE); repetition time/echo time 28.5/7.9 msec; inversion time 65 msec; 31.2 kHz, flip angle 30°, four excitations; 40 slice-encoding steps; scan time 7 min 36 seconds] with chemical shift fat-suppression was used 10, 20, 30, and 45 min after injection of the contrast agents. The coronal images were reconstructed with 0.6-mm section thickness at every 0.3-mm. The FOV was 6×3 cm and the size of the matrix was 512×256. The intensities of the regions of interest (ROIs) (for example, a whole axillary lymph node, and the liver) were measured, and then the time-intensity curves analyzed. The data were expressed as the axillary lymph node-to-muscle and the axillary lymph node-to-liver ratios. The slice data were processed into 3D images using the MIP method with the same window and level (window 3500 and level 2300) (Advantage Windows, General Electric Medical System). Two board-certified radiologists separately read a set of stereo views of 3D-MRL for all studies, and estimated the visualization of the thoracic duct. After imaging, the mice were sacrificed by carbon dioxide inhalation.

Enlarged lymph nodes that clearly contained abnormal lesions, as demonstrated by MRL, were isolated from the mouse following MRL image acquisition using flow cytometry. After the lymphocytes were purified by Ficoll density separation, the cells were first incubated with an anti-CD16 antibody (Pharmingen, San Diego, Calif.) to block FcγR-mediated staining, and then stained with a combination of FITC-anti CD3 (Pharmingen), Phycoerythrin-NK1.1 (Pharmingen), and Cychrome-anti CD8 (Pharmingen). They were incubated for 15 minutes at room temperature.

Statistical analyses were performed using either Student's t-test or a one-way analysis of variance (ANOVA), with a pairwise comparison using the Bonferroni method for signal intensity curves (Statview; SAS Institute Inc., Cary, N.C.).

### IV Results

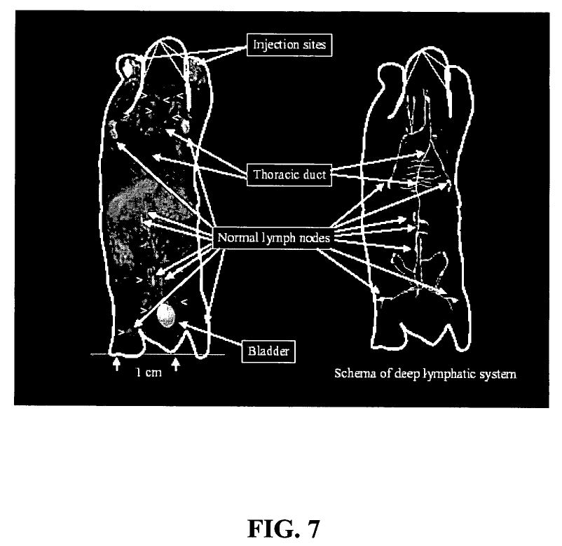

As shown in FIG. 7, the PAMAM-G8 contrast agent enabled most of the deep lymph nodes to be visualized in a mouse. The schema shown in FIG. 7 shows that a number of normal lymph nodes and the thoracic duct were imaged following injection of the PAMAM-G8 contrast agent intradermally into the extremities of the mouse.

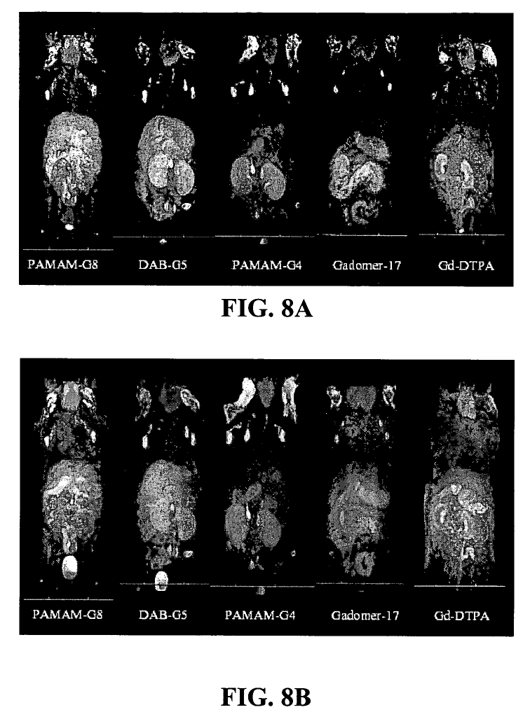

A comparison of the images obtained using the PAMAM-G8, PAMAM-G4, and DAB-G5 contrast agents to images obtained using Gadomer-17 and GPDM is shown in FIG. 8. FIG. 8A compares the images obtained using these agents 10 minutes after injection. The lymph nodes (particularly those about ⅔ of the way up the body of the mice) appear much brighter and well-defined in the images obtained using the dendrimer conjugates than in the images obtained with Gadomer-17 and GPDM. As shown in FIG. 8B, the dendrimer conjugates exhibit persistent superior image contrast and detail of the lymphatic system 45 minutes after injection in comparison to Gadomer-17 and GPDM. Virtually no image enhancement is observed after 45 minutes using GPDM. Most of the deep lymph nodes were visualized throughout the mouse at all time points examined using the dendrimer conjugates. Gadomer-17 allowed visualization of the deep lymph nodes, albeit not as clearly and brightly as the dendrimer-based agents. In contrast, GPDM did not allow most of the lymph nodes to be visualized even at early times.

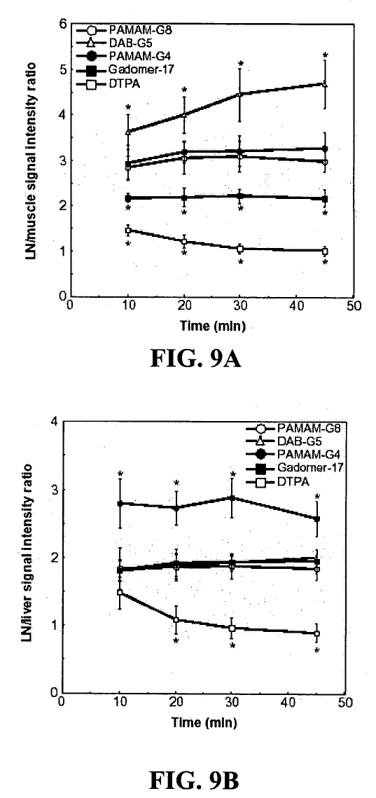

To compare the clarity of the images with different contrast agents in a semi-quantitative fashion, the ratio between the intensity of signals (T₁-weighted signal) obtained from the axillary lymph node and that from the neighboring muscle tissue (the “background”) was calculated at different times following administration. The results are shown in FIG. 9A. The axillary lymph node-to-background ratio obtained with DAB-G5 was significantly higher than that obtained with either PAMAM-G8 or PAMAM-G4 at all time points examined (P<0.01). The axillary lymph node-to-background ratio obtained with PAMAM-G8 or PAMAM-G4 was significantly higher than that measured with Gadomer-17 and GPDM at all time points examined. Furthermore, the axillary lymph node-to-background ratio obtained with Gadomer-17 was significantly higher than that acquired with GPDM (P<0.01). These results illustrate the superior image quality obtained with a number of the disclosed dendrimer conjugates.

To further compare the ability of the five contrast agents to aid visualization (clarity of contrast) of lymph nodes close to the organs responsible for the excretion of the contrast agents, signal intensity ratios were then measured between the signal at the axillary lymph node and the signal at the liver (FIG. 9B). The axillary lymph node-to-liver ratio obtained with PAMAM-G4 was significantly higher than that acquired with PAMAM-G8, DAB G-5, or Gadomer-17 at all time points examined (P<0.01). The axillary lymph node-to-liver ratios of PAMAM-G8, DAB-G5, or Gadomer-17 were nearly equivalent, but were significantly higher than that measured with GPDM (P<0.01).

The lymphatic vessels were better visualized with PAMAM-G8 compared to all other agents examined, followed by the PAMAM-G4 agent. In particular the PAMAM-G8 contrast agent permitted visualization by a radiologist of the thoracic duct in all mice at all times after administration. The ability of the contrast agents used in this Example to provide sufficient contrast to allow identification by radiologists of the thoracic duct are summarized in Table 2 below.

TABLE 2
Visualization of Thoracic Duct on the Dynamic Study of MRL (Number
of Mice in Which the Thoracic Duct was Visualized/Number
of Mice Examined)

Time (min)

| | 10 | 20 | 30 | 45 |
|---|---|---|---|---|
| PAMAM-G8/Normal Mice | 10/10 | 10/10 | 10/10 | 10/10 |
| DAB-G5/Normal Mice | 3/5 | 2/5 | 0/5 | 0/5 |
| PAMAM-G4/Normal Mice | 4/5 | 4/5 | 3/5 | 2/5 |
| Gadomer-17/Normal Mice | 2/5 | 2/5 | 1/5 | 1/5 |
| GPDM/Normal Mice | 2/5 | 0/5 | 0/5 | 0/5 |

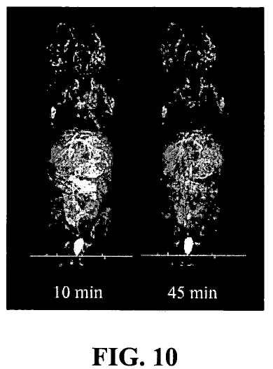

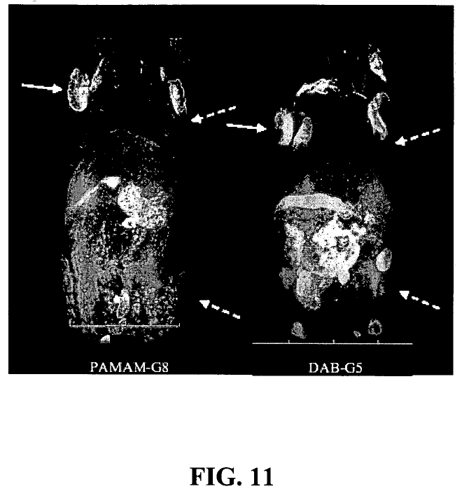

The five contrast agents were next evaluated for their ability to visualize the status of diseases associated with the lymphatic system in three different mouse models. Lymphangitis was induced in mice by injecting Conocanavin A intravenously, as typically accompanies systemic dilatation of lymphatic vessels. As shown in FIG. 10, dynamic 3D-micro-MRL with PAMAM-G8 demonstrated the remarkable dilation of the lymphatic vessels throughout the body, especially in the liver. Enhanced structures were mostly shown by 2D-micro-MRL along vessels, including hepatic veins (data not shown). Those structures correlated well with the dilated lymphatic vessels on histological specimens.

Mice with lymphoproliferation/lymphoma were also examined. The IL-15 transgenic mice that were used develop manifested polyclonal expansion of NK, NK-T, γδ-T, and memory phenotype CD8⁺ T-cells in the periphery, and macroscopic examination demonstrated enlarged spleen and lymph nodes (data not shown). Dynamic 3D-micro-MRL demonstrated considerable multiple lymph node swellings with central filling defects (non-enhancing area), when either PAMAM-G8 or DAB-G5 was used as the contrast agent (left and right images, respectively). In these images, taken 45 minutes after injection of the respective contrast agents, dilation of subcutaneous lymphatic vessels (broken arrows) and swollen right axillary lymph nodes (solid arrows) are indicated. As seen, however, dilation of lymphatic vessels in these mice was best visualized with PAMAM-G8 (left image). Pathological examination of the swollen lymph nodes showing filling defects yielded results consistent with the micro-MRL observations. In brief, an accumulation of homogenous, dense lymphoid cells in major lymph nodes from aged (>32 wk) IL-15 transgenic mice were observed, indicating that these mice manifested a chronic lymphoproliferative status. In particular, the germinal center structure was no longer seen in these lymph nodes. Furthermore, immunological examination revealed that these cells were mostly mature CD8⁺ T-cells. The densely packed infiltrating CD8⁺ T-cells within the lymph node appeared to form a tight boundary, preventing free penetration of the macromolecular contrast agent. Although polyclonal expansion of NK, NK-T, and γδ-T IELs lymphocytes were observed in the IL-15 transgenic mouse, these cells did not manifest chronic expansion or infiltration into the lymph nodes. These observations collectively demonstrate that the IL-15 transgenic mice develop chronic CD8⁺ T cell expansion/proliferation in multiple lymph nodes with aging, which may lead to the onset of a lethal pathological condition (such as lymphoma).

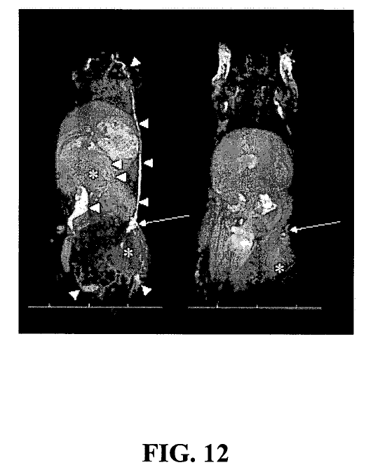

The potential of 3D-MRL with PAMAM-G8 to examine lymph nodes infiltrated with non-lymphoid cells also was examined. MC-38 colorectal cancer cells form metastatic growths in lymph nodes of syngenic C57BL6 mice following intravenous injection, and thus provide an appropriate model in which to evaluate this system. A 3D image of a mouse with a lymph node metastasis obtained 45 minutes after injection of the PAMAM-G8 agent is shown in FIG. 12 (left image). Large inguinal and abdominal tumors (asterisks) accompanied by the left inguinal lymph node (long arrow) are seen, along with dilated lymphatic vessels surrounding the tumor and a collateral lymphatic vessel, which communicated with the thoracic duct via the axillary lymph node (arrowheads). In contrast, mice with a subcutaneously xenographed MC-38 tumor in the same location did not show any abnormalities in either the lymph nodes or the lymphatic vessels by 3D-MRL with PAMAM-G8 (right image), confirming that the growth of tumor cells in the lymph node tissue specifically caused the abnormal image characteristics visualized by this method.

The effectiveness of the disclosed methods in diagnosing and differentiating various lymphatic disease by visualizing abnormally developing lymph nodes and lymphatic vessels associated with inflammation, proliferative disorder, and tumor metastasis were demonstrated in this example. In addition, each of the dendrimer-based contrast agents exhibited distinct characteristics, which may be exploited for different purposes in clinical applications. For example, PAMAM-G8 appears to be better suited for imaging of lymphatic vessels and diverse other components of the lymphatic system, whereas DAB-G5 may be better suited for imaging of lymph nodes. PAMAM-G4 appears to be particularly suited for visualization of abdominal lymph nodes adjacent to the liver based on its high lymph node to liver signal intensity.

In summary, the disclosed dendrimer conjugates are superior contrast agents for imaging the lymphatic system in comparison to Gadomer-17 and GPDM, even at lower dosages warranted by the potential toxicity of metal ions, such as gadolinium ions, that may be released from the disclosed dendrimer conjugates.

### EXAMPLE 3

### Comparison of PAMAM-G8 and GPDM for Detecting and Differentiating Lymphatic Disorders Including Infection and Metastatic Conditions

This example further demonstrates the ability of the disclosed methods to assess and differentiate differing disease states of the lymphatic system. In this example, the PAMAM-G8 contrast agent is compared to GPDM. PAMAM-G8 was prepared as described in Example 2, and GPDM was purchased (Schering AG, Berlin, Germany). Animal models, administration of the contrast agents, and 3D-micro-MRL were as described in Example 2.

### I. Results

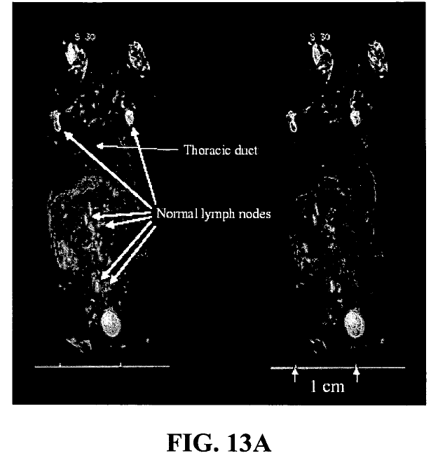

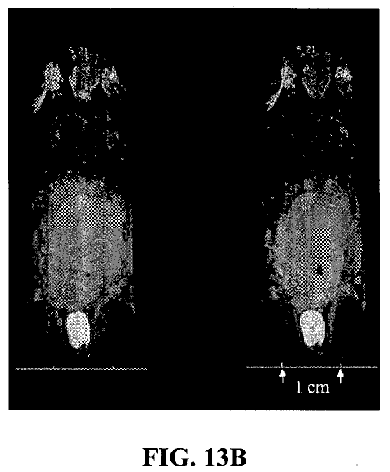

As seen in FIG. 13, most deep lymph nodes throughout the body were visualized by 3D-microMR-lymphangiography with PAMAM-G8 (FIG. 13A), but not with GPDM (FIG. 13B). In addition, the thoracic duct was visualized in all six mice that were given injections of PAMAM-G8, but not in mice given injections of GPDM. Other lymphatic vessels were also better visualized with PAMAM-G8 than with GPDM.

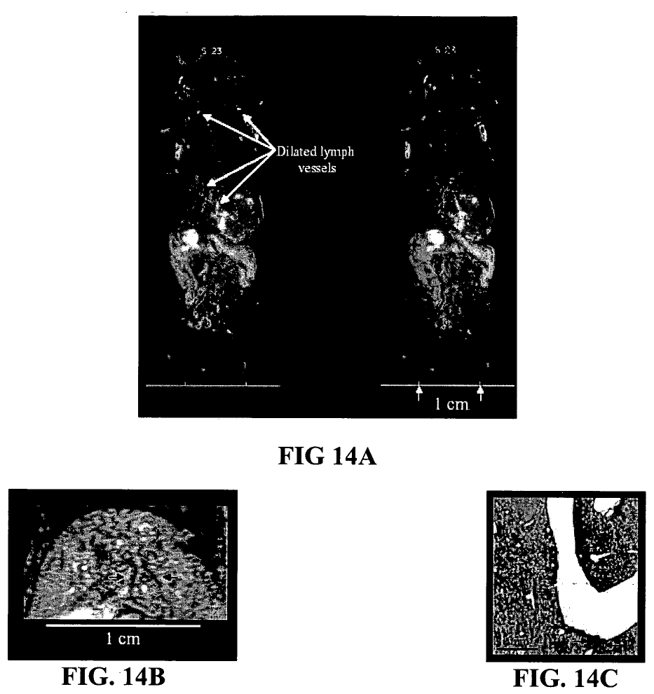

Concanavilin A was injected into normal mice to induce lymphangitis. As shown in FIG. 14A, three-dimensional micro-MRL using PAMAM-G8 was able to detect remarkable dilation of lymphatic vessels (arrows) throughout the whole body, and especially in the liver. The enhancement in the liver was seen adjacent to the vascular structure, but the vascular structures themselves did not show enhancement (arrows in FIG. 14B). Histology analysis (FIG. 14C) revealed that the lymphocytes had mainly infiltrated adjacent to the vascular structures with cavernous dilation (>10 μm) of lymphatic ducts filled with massive lymphocytes (arrows), corresponding to the enhancement location and consistent with the imaging results.

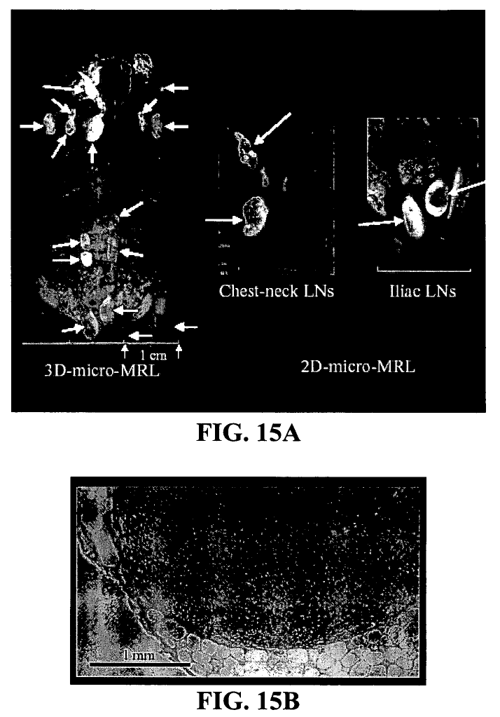

Lymph node changes in a proliferative or neoplastic model also were evaluated. FIG. 15A shows a series of images taken of a IL-15 transgenic mice which showed considerable lymph node swelling (3D image) with non-enhancing central filling defects (2D-images). The abnormal lymph nodes identified by micro-MRL were targeted for removal, living cell sampling and subsequent analysis. Immunological and molecular biological analyses to demonstrate the cellular phenotypes, the receptor expressions, and the clonality of the infiltrative cells in individual mice also were performed. These pathological examinations confirmed that the observation of filling defects in the images were due to replacement of the germinal center structure of lymph nodes by a homogeneous dense infiltration of lymphoid cells that restricted access of the contrast agent (as shown in the histological image of FIG. 15B). These observations collectively demonstrate that with age the IL-15 transgenic mouse develops CD8⁺ T-cell expansion and proliferation in multiple lymph nodes, which may lead to the onset of lethal pathological conditions such as lymphoma.

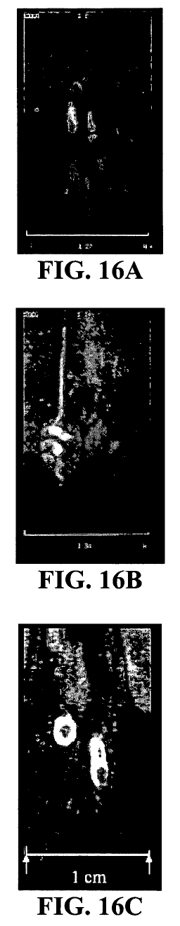

Nude mice that develop spontaneous oral ulcers and urinary tract infections also were examined. Images taken 45 minutes after injection with PAMAM-G8 in this infection model, showed an irregular dilation of the lymphatic vessels in the lymph nodes (FIG. 16B). These enlarged infections lymph nodes can be differentiated in images from normal lymph nodes (FIG. 16A, also obtained with PAMAM-G8), in which small lymph nodes are observed, and from enlarged lymph nodes in the metastatic IL-15 transgenic mouse model (FIG. 16C, also obtained with PAMAM-G8), which exhibit central filing defects characteristic of the presence of metastatic cells in the lymph node.

The MRL methods described herein are applicable to both investigative studies in laboratory animals and in clinical practice with human subjects. With respect to micro-MRL in mice, the disclosed methods permit detection of abnormalities in the lymphatic system throughout the whole body in a live animal, allowing evaluation of time-dependent changes in the same mouse (data not shown). The methods also permitted targeted removal of and subsequent analysis of involved lymph nodes in IL-15 transgenic mice with lymphoadenopathy. Since immunological and molecular biological analyses demonstrated the cellular phenotypes, the receptor express, and the clonality of the infiltrative cells, the results will be diagnostically useful in determining the consequences of the expantion of CD8⁺ T-lymphocytes in IL-15 transgenic mice.

The dilated liver and mesenteric lymphatic systems were enhanced and visualized in the concanavalin A-induced lymphangitis model by this method. The liver lymphatic enhancement was found just surrounding the vasculature in the disease model mice. The enhancement tended to locate along the hepatic veins as shown in FIG. 14. Therefore, an amount of contrast agent which attached to lymphocytes, could migrate from the main trunk of the lymphatic vessels back to liver or mesenteric systems associated with lymphocyte infiltration. Thus, it might enhance the liver and mesenteric system lymphatic systems, especially under the condition of lymphatic congestion.

In conclusion, micro-MRL with the PAMAM-G8 contrast agent was able to visualize most of the lymph nodes throughout the body and could distinguish infections expansion of lymphocytes from that caused by chronic lymphoproliferative conditions.

### EXAMPLE 4

### MRI

MRI is a technique that allows whole body in vivo imaging in three dimensions at high resolution. In MRI, a static magnetic field is applied to the object of interest while simultaneously or subsequently applying pulses of radio frequency (RF) to change the distribution of the magnetic moments of protons in the object. The change in distribution of the magnetic moments of protons in the object from their equilibrium (normal) distribution to a non-equilibrium distribution and back to the normal distribution (via relaxation processes) constitute the MRI signal.

The longitudinal relaxation time, T₁, is defined as the time constant of the exponential recovery of proton spins to their equilibrium distribution along an applied magnetic field after a disturbance (e.g. a RF pulse). The transverse relaxation time, T₂, is the time constant that describes the exponential loss of magnetization in a plane transverse to the direction of the applied magnetic field, following a RF pulse that rotates the aligned magnetization into the transverse plane. Magnetic resonance (MR) contrast agents assist this return to a normal distribution by shortening T₁ and/or T₂ relaxation times.

Signal intensity in biological MRI depends largely on the local value of the longitudinal relaxation rate (1/T₁), and the transverse relaxation rate (1/T₂) of water protons. Contrast agents will increase 1/T₁ and/or 1/T₂, depending on the nature of the agent and the strength of the applied field. MRI pulse sequences that emphasize changes in 1/T₁ are referred to as T₁-weighted and those that emphasize changes in 1/T₂ are referred to as T₂-weighted. MR contrast agents that include gadolinium (III) ions increase both 1/T₁ and 1/T₂, and are primarily used with T₁-weighted imaging sequences, since the relative change in 1/T₁ in tissue is typically much greater than the change in 1/T₂. Iron particles, by contrast, provide larger relative changes in 1/T₂, and are best visualized in a T₂-weighted image.

Advances in MRI have tended to favor T₁ agents such as gadolinium (III) based contrast agents. Faster scans with higher resolution require more rapid RF pulsing, and can lead to loss of the MRI signal through saturation effects. T₁ agents relieve this saturation and restore signal intensity by stimulating relaxation of nuclear spins between RF pulses. Furthermore, T₁ agents are compatible with image guided surgical procedures such as needle biopsy, as objects inserted into a subject's body will appear in a T₁ image.

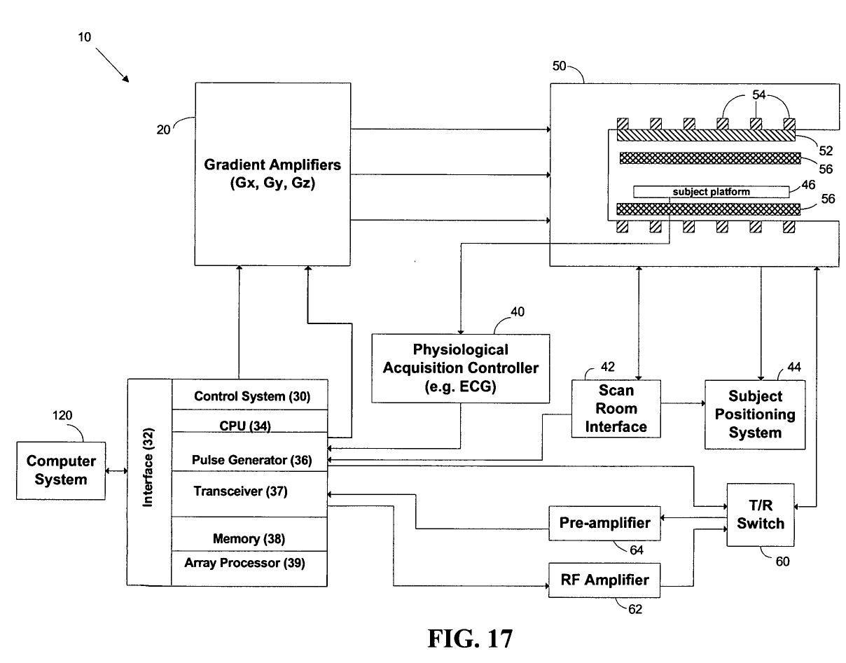

An exemplary MRI system is illustrated in FIG. 17. Referring to FIG. 17, the major components of a MRI system 10 that may be used to practice the disclosed methods are shown. The operation of the system is controlled by computer system 120. The computer system 120 includes a number of modules that communicate with each other, and with control system 30, through interface 32.

The control system 30 includes a set of modules connected together by an interface 32, and also connected to computer system 120 through interface 32. These modules include a CPU module 34. A pulse generator module 36 operates the system components to carry out the desired scan sequence and produces data which indicates the timing, strength and shape of the RF pulses produced, and the timing and length of the data acquisition window. The pulse generator module 36 connects to a set of gradient amplifiers 20, to indicate the timing and shape of the gradient pulses that are produced during the scan. The pulse generator module 36 also receives subject data from a physiological acquisition controller 40 that receives a signal from one or more sensors connected to the subject, such as an ECG signal from electrodes attached to the subject. The pulse generator module 36 also connects to a scan room interface circuit 42 that receives signals from various sensors associated with the condition of the patient and the magnet system. It is also through the scan room interface circuit 42 that a subject positioning system 44 receives commands to move the subject on subject platform 46 to the desired position for the scan.

The gradient waveforms produced by the pulse generator module 36 are applied to the gradient amplifier system 20 having Gx, Gy, and Gz amplifiers. Each gradient amplifier excites a corresponding gradient coil in an assembly designated 52. The gradient coil assembly 52 forms part of a magnet assembly 50 which includes a polarizing magnet 54 and a whole-body RF coil 56. Although not shown, additional coils may be used to provide more detailed images of a particular anatomical location within or on a subject. For example an external coil such as a breast coil, head coil, cardiac coil, CTL coil, shoulder coil, or torso-pelvis coil is used (these types of coils and others are available from GE Medical Systems, Milwaukee, Wis.). In a particular embodiment, a breast coil is located over a female subject's mammary glands to provide more detailed images of the mammary tissue. A transceiver module 37 in the control system 30 produces pulses that are amplified by a RF amplifier 62 and coupled to the RF coil 56 by a transmit/receive switch 60. The resulting signals radiated by the excited nuclei in the patient may be sensed by the same RF coil 56 and coupled through the transmit/receive switch 60 to a preamplifier 64. The amplified NMR signals are demodulated, filtered, and digitized in the receiver section of the transceiver 37. The transmit/receive switch 60 is controlled by a signal from the pulse generator module 36 to electrically connect the RF amplifier 62 to the coil 56 during the transmit mode and to connect the preamplifier 64 during the receive mode. The transmit/receive switch 60 also enables a separate RF coil (for example, a surface coil) to be used in either the transmit or receive mode.

The following is a brief description of the acquisition and storage of MR data. The NMR signals picked up by the RF coil 56 are digitized by the transceiver module 37 and transferred to a memory module 38 in the system control 32. When a scan is completed, an array of raw k-space data has been acquired in the memory module 38. This raw k-space data is rearranged into separate k-space data arrays for each image to be reconstructed, and each of these is input to an array processor 39 which operates to Fourier transform the data into an array of image data. This image data is conveyed through interface 32 to the computer system 120, where it may be stored and/or further processed using methods known to those skilled in the art.

### EXAMPLE 5

### Dendrimer Conjugates

Dendrimer-based contrast agents may be prepared by reacting a surface group of a dendrimer with the reactive group of a bifuictional chelating agent and then reacting the metal chelating group of the bifunctional chelating agent with a metal ion. Alternatively, a metal ion is reacted with the metal chelating group of the bifunctional chelating agent prior to reacting the reactive group of the bifunctional chelating agent with a surface groups of the dendrimer. Metal chelation is typically carried out in solution, and desirably avoids the use of strong acids or bases. In particular embodiments, a dendrimer, such as DAB-G4D, DAB-G5D, DAB-G6D, DAB-G7D, DAB-G8D, PAMAM-G4D, PAMAM-G5D, PAMAM G6D, PAMAM-G7D, or PAMAM-G8D is reacted with 1B4M and gadolinium ions (in either order as discussed below) to provide dendrimer conjugates suitable for lymphatic system imaging.

Thus, in one aspect, dendrimer conjugates suitable for lymphatic system imaging include DAB-G4, DAB-G5, DAB-G6, DAB-G7, DAB-G8, PAMAM-G4, PAMAM-G5, PAMAM-G6, PAMAM-G7, and PAMAM-G8, which all are 1B4M conjugates with chelated Gd³⁺ ions. In more particular examples, dendrimer conjugates for use as lymphatic system contrast agents include DAB-G5, PAMAM-G6 and PAMAM-G8. In still more particular examples, a PAMAM-G6 dendrimer conjugate is used for lymphatic system imaging.

Table 1 compares some properties of some particular dendrimer conjugates, Gadomer-17 and the simple gadolinium chelate GPDM.

TABLE 3
Comparison of Example Contrast Agents

| | | | Approximate | |
|---|---|---|---|---|
| Contrast | Approximate | | Core MW | |
| Agent | MW (kD) | Gd atoms | (kD) | Dendrimer Type |
| PAMAM-G4 | 58 | 64 | 14.2 | PAMAM |
| PAMAM-G5 | 117 | 128 | 29 | PAMAM |
| PAMAM-G6 | 235 | 256 | 58 | PAMAM |
| PAMAM-G7 | 470 | 512 | 116 | PAMAM |
| PAMAM-G8 | 960 | 1024 | 233 | PAMAM |
| DAB-G5 | 51 | 128 | n/a | DAB |
| Gadomer-17 | 30 | 24 | n/a | Aromatic Ring |
| | | | | Core Dendrimer |
| Gd-DTPA- | 0.94 | 1 | n/a | n/a |
| dimeglumine | | | | |

n/a - not available or not applicable

The disclosed dendrimer conjugates exhibit a range of properties that permit detailed and selective imaging of particular components (or finctions) of the lymphatic system (such as lymphatic vessels, lymph nodes and flow of lymphatic fluid). For example, PAMAM-G8 exhibits lymphotropic behavior (accumulation in the lymph system) and minimal leakage out of the lymphatic vessels, which aids in the visualization of both thick and thin lymphatic vessels. In contrast, PAMAM-G4 and DAB-G5 tend to accumulate in the lymph nodes rather than the vessels, and provides detailed visualization of these structures. PAMAM-G4 has a short survival in the blood circulation due to a rapid renal excretion without significant retention in other organs. PAMAM-G6 has an intermediate survival period in the lymph system, and is particularly suitable for dynamic imaging of the lymph system (for example, for following lymphatic flow).

Additional dendrimers may be used to provide dendrimer conjugates that can be utilized in the disclosed methods. For example, polyakylenimine dendrimers and PAMAM dendrimers having different initiator cores, but similar molecular weights (within about 25%, for example within 15%, 10% or 5% of the MW) to those dendrimers specifically disclosed may be utilized. Such dendrimers also may be synthesized according to the methods disclosed in Womer and Mulhaupt, Angewandte Chemie, Int. Ed., 32: 1306-1308, 1993. De Brabander-van den Berg and Meijer describe similar methods, and in particular, methods for making polypropylenimine dendrimers having various initiator cores, such as ammonia, ethylenediamine, propylenediamine, diaminobutane and other polyamines such as tris-aminoethylamine, cyclene, hexaazacyclooctadecane, 1,5 diaminopentane, ethylenetriamine, triethylenetetramine, 1,4,8,11-tetraazaundecane, 1,5,8,12-tetraazaundodecane, and 1,5,9,13-tetraazatridecan (De Brabander-van den Berg and Meijer, Angewandte Chemie, Int. Ed., 32: 1308, 1993). Typically, the surface of the polypropylenimine dendrimer will have one or more amino groups. However, some or all of the surface amino groups may be modified, for example, to provide other reactive groups or charged, hydrophilic, and/or hydrophobic groups such as carboxylate, hydroxyl and alkyl groups on the surface. Similar schemes may be used to synthesize polybutylenimine and higher polyalkylenimine dendrimers. Additional information regarding the synthesis of a variety of dendrimers with branches formed from vinyl cyanide units is provided in PCT Publication WO 93/14147.

PAMAM dendrimers also may be synthesized from a variety of core molecules (e.g., those described above for DAB dendrimers) according to the methods disclosed in U.S. Pat. No. 5,338,532. Dendrimers having other surface groups, such as carboxylate and hydroxyl, also are available commercially (Aldrich, Milwaukee, Wis.) or may be provided by the methods disclosed in U.S. Pat. No. 5,338,532.

The metal chelate in a dendrimer conjugate is a complex of a metal ion and a metal chelating group (a group of atoms that serves to bind the metal ion). Examples of metal chelating groups include natural and synthetic amines, porphyrins, aminocarboxylic acids, iminocarboxylic acids, ethers, thiols, phenols, glycols and alcohols, polyamines, polyaminocarboxylic acids, polyiminocarboxylic acids, aminopolycarboxylic acids, iminopolycarboxylic acids, nitrilocarboxylic acids, dinitrilopolycarboxlic acids, polynitrilopolycarboxylic acids, ethylenediaminetetracetates, diethylenetriaminepenta or tetraacetates, polyethers, polythiols, cryptands, polyetherphenolates, polyetherthiols, ethers of thioglycols or alcohols, polyaminephenols, all either acyclic, macrocyclic, cyclic, macrobicyclic or polycyclic, or other similar ligands which produce stable metal chelates or cryprates (including sepulchrates, sacrophagines, and crown ethers).

Specific examples of metal chelating groups include diethylenetriaminepentaacetic acid (DTPA), 1,4,7,10-tetraazacyclododecanetetraacetic acid (DOTA), 1,4,7,10-tetraazacyclododecane-1,4,7-triacetic acid (DO3A), 1-oxa-4,7,10-triazacyclododecane-triacetic acid (DOXA), 1,4,7-triazacyclononanetriacetic acid (NOTA), 1,4,8,11 -tetraazacyclotetradecanetetraacetic acid (TETA), DOTA-N(2-aminoethyl)amide and DOTA-N-(2-aminophenethyl)amide, BOPTA, HP-DO3A, DO3MA, 1B4M and various derivatives and combinations thereof. Additional examples of metal chelating groups have been described by Caravan et al. (Caravan et al., Chem. Rev. 99: 2293-2352, 1999). Since release of metal ions from such chelating groups can be dangerous to a subject, it is advantageous to select a metal chelating group that tightly binds a metal ion. Therefore, a high stability constant for the metal chelate is desired.

The reactive group of a bifunctional chelating agent is a group of atoms that that will undergo a reaction with a surface group of a dendrimer to form a bond, such as a covalent bond. Examples of reactive groups include carboxylic acid groups, diazotiazable amine groups, N-hydroxysuccinimidyl, esters, aldehydes, ketones, anhydrides, mixed anhydrides, acyl halides, maleimides, hydrazines, benzimidates, nitrenes, isothiocyanates, azides, sulfonamides, bromoacetamides, iodocetamides, carbodiimides, sulfonylchlorides, hydroxides, thioglycols, or any reactive group known in the art as usefuil for forming conjugates. If the dendrimer is a DAB-Am dendrimer, the reactive group may be a finctional group capable of undergoing reaction with an amino group of the DAB-Am dendrimer.

Specific examples of bifinctional chelating agents include bifinctional diethylenetriaminepentaacetic acid (DTPA) derivatives such as those disclosed in U.S. Pat. No. 5,434,287 to Gansow et al. Other examples include polysubstituted diethylenetriaminepentaacetic acid chelates such as those described by Gansow et al. in U.S. Pat. No. 5,246,692. Bifinctional chelating agents comprising 1,4,7,10-Tetraazacyclododecane-N,N′,N″,N′″-tetraacetic acid (DOTA) and its derivatives are also useful. Examples of bifunctional DOTA derivatives are provided in U.S. Pat. No. 5,428,154 to Gansow et al. and references therein. A particular example of a bifunctional chelating agent is 2-(p-isothiocyanatobenzyl)-6-methyl-diethylenetriaminepentaacetic acid (1B4M).

Additional examples of bifinctional chelating agents and metal chelating groups may be found in U.S. Pat. Nos. 5,292,868, 5,364,613, 5,759,518, 5,834,020, 5,874,061, 5,914,095, 5,958,373, 6,045,776, 6,274,713; PCT Publications WO 95/17451 and WO 95/09564; U.S. Patent Application Publication US2002/0004032; European Patent Application EP 0882454; and European Patent Specifications EP 0416033 and EP 0497926.

Metals ions of the metal chelates may be paramagnetic ions if the imaging agent is to be used as a MRI contrast agent. Suitable ions include ions of metals having atomic numbers of 22-29 (inclusive), 42, 44 and 58-70 (inclusive) and combinations thereof. In particular embodiments, the metal ions have an oxidation state of 2 or 3. Examples of such metal ions are chromium (III), manganese (II), iron (II), iron (III), cobalt (II), nickel (II), copper (II), praseodymium (III), neodymium (III), samarium (III), gadolinium (III), terbium (III), dysprosium (III), holmium (III), erbium (HII) and ytterbium (III), and combinations thereof. Particular examples of useful ions for MRI include the paramagnetic ions of gadolinium, dysprosium, cobalt, manganese, and iron. In a particular disclosed embodiment, the metal ion is a Gd (III) ion.

If the macromolecular imaging agent is to be used as an X-ray contrast agent (such as for CT), the metal ion may be selected from the ions of W, Bi, Hg, Os, Pb, Zr, lanthanides, and combinations thereof. If a combined MlI/X-ray contrast agent is desired, the metal ion may be selected from the paramagnetic lanthanide ions. If a scintographic imaging agent is desired, the metal may be radioactive, such as the radioactive isotopes of In, Tc, Y, Re, Pb, Cu, Ga, Sm, Fe, or Co.

In some embodiments, the methods include administering a dendrimer conjugate to a subject where the metal chelating group of the dendrimer conjugate is diethylenetriaminepentaacetic acid (DTPA), 1,4,7,10-tetraazacyclododecanetetraacetic acid (DOTA), 1,4,7,10-tetraazacyclododecane-1,4,7-triacetic acid (DO3A), 1-oxa-4,7,10-triazacyclododecane-triacetic acid (DOXA), 1,4,7-triazacyclononanetriacetic acid (NOTA), 1,4,8,11 -tetraazacyclotetradecanetetraacetic acid (TETA), DOTA-N-(2-aminoethyl)amide and DOTA-N-(2-aminophenethyl)amide, BOPTA, HP-DO3A, DO3MA, 2-(p-isothiocyanatobenzyl)-6-methyl-diethylenetriaminepentaacetic acid (1B4M), or derivatives and combinations thereof. The metal chelate may comprise an ion of a metal having an atomic number of 22-29, 42, 44, 58-70 or combinations thereof. In particular embodiments, the ion is a chromium (III) ion, manganese (II) ion, iron (II) ion, iron (III) ion, cobalt (II) ion, nickel (II) ion, copper (II) ion, praseodymium (III) ion, neodymium (III) ion, samarium (III) ion, gadolinium (III) ion, terbium (III) ion, dysprosium (III) ion, holmium (III) ion, erbium (III) ion, ytterbium (III) ion or a combination of such ions. In particular embodiments, the dendrimer conjugate is a Gd-1B4M conjugate and is DAB-G5, DAB-G6, DAB-G7, DAB-G8, PAMAM-G4, PAMAM-G5, PAMAM G6, PAMAM-G7 or PAMAM-G8.

1. A method for lymphatic system imaging, comprising:
administering an image enhancing amount of a dendrimer conjugate to a subject, the dendrimer conjugate comprising a DAB-G4D, DAB-G5D, DAB-G6D, DAB-G7D, DAB-G8D, PAMAM-G4D, PAMAM-G5D, PAMAM-G6D, PAMAM-G7D, or PAMAM-G8D dendrimer and a metal chelate; and
detecting a difference in an image signal intensity of at least a portion of the lymphatic system after the dendrimer conjugate is administered, wherein the difference in image signal intensity indicates a condition of the lymphatic system.

2. The method of claim 1, wherein the dendrimer is DAB-G5D, PAMAM-G4D, PAMAM-G6D or PAMAM-G8D.

3. The method of claim 2, wherein the dendrimer is PAMAM-G6D.

4. The method of claim 1, wherein the metal chelate is a DTPA metal chelate, a DOTA metal chelate, a DO3A metal chelate, a DOXA metal chelate, a NOTA metal chelate, a TETA metal chelate, a DOTA-N(2-aminoethyl)amide metal chelate, a DOTA-N-(2-aminophenethyl)aniide metal chelate, a BOPTA metal chelate, a HP-DO3A metal chelate, a DO3MA metal chelate, or a 1 B4M metal chelate.

5. The method of claim 1, wherein the metal chelate comprises a metal ion of a metal selected from the metals having atomic numbers of 22-29, 42, 44 and 58-70 and combinations thereof.

6. The method of claim 5, wherein the image signal intensity is a magnetic resonance signal intensity and the metal ion is chromium (III), manganese (II), iron (II), iron (III), cobalt (II), nickel (II), copper (II), praseodymium (III), neodymium (III), samarium (III), gadolinium (III), terbium (III), dysprosium (III), holmium (III), erbium (III) or ytterbium (III).

7. The method of claim 6, wherein the metal ion is gadolinium (III).

8. The method of claim 7, wherein the metal chelate is a 1B4M metal chelate and the dendrimer conjugate is DAB-G4, DAB-G5, DAB-G6, DAB-G7, DAB-G8, PAMAM-G4, PAMAM-G5, PAMAM-G6, PAMAM-G7 or PAMAM-G8.

9. The method of claim 8, wherein the dendrimer conjugate is DAB-G5, PAMAM-G4, PAMAM-G6, or PAMAM-G8.

10. The method of claim 9, wherein the dendrimer conjugate is PAMAM-G6.

11. The method of claim 1, wherein the dendrimer conjugate further comprises an optical or fluorescent moiety.

12. The method of claim 1, wherein administering an imaging enhancing amount of the dendrimer conjugate comprises administering a dose between about 0.0001 mmol metal/kg of the subject's body weight and about 1.0 mmol metal/kg of the subject's body weight.

13. The method of claim 1, wherein administering comprises injecting the dendrimer conjugate intravenously, intracutaneously, intradermally, subdermally, peritonially, intratumorally, into the lymphatic system, into a surgical field or peritumorally.

14. The method of claim 13, wherein administering the dendrimer conjugate comprises injecting the dendrimer conjugate intratumorally or peritumorally.

15. The method of claim 1, wherein the portion of the lymphatic system comprises a lymph node.

16. The method of claim 15, wherein detecting the difference comprises detecting a lack of image enhancement in the lymph node as an indication of the presence of metastatic cancer cells in the lymph node.

17. The method of claim 15, wherein the dendrimer conjugate is DAB-G5 or PAMAM-G4.

18. The method of claim 1, wherein the portion of the lymphatic system comprises a lymphatic vessel.

19. The method of claim 18, wherein detecting the differences comprises detecting an irregular enhancement as an indication of infection.

20. The method of claim 1 fuirther comprising detecting differences in signal intensity of the portion of the lymphatic system over time to detect lymphatic flow through the portion of the lymphatic system.

21. The method of claim 18, wherein the dendrimer conjugate is PAMAM-G8.

22. The method of claim 1, wherein the portion of the lymphatic system is an axillary lymph node, an inguinal lymph node, a thoracic duct, a subcutaneous lymph node, a deep lymph node or a deep lymphatic vessel.

23. A method for identifying a lymph node into which lymph fluid flows from a tumor in a subject, comprising:
administering an image-enhancing amount of a dendrimer conjugate to an intratumoral or peritumoral site of administration of the subject, where the dendrimer conjugate is DAB-G4, DAB-G5, DAB-G6, DAB-G7, DAB-G8, PAMAM-G4, PAMAM-G5, PAMAM-G6, PAMAM-G7 or PAMAM-G8;
imaging a path of lymph fluid flow from the site of administration using magnetic resonance imaging of the dendrimer conjugate to provide an image of the lymphatic system draining the tumor; and
identifying the lymph node in the image of the lymphatic system draining the tumor.

24. The method of claim 23, wherein the lymph node is a first lymph node along the path of lymph fluid flow.

25. The method of claim 24 further comprising imaging the path of lymph flow over time to locate a lymph node into which lymph fluid flowing from the tumor first appears.

26. The method of claim 23, further comprising detecting metastatic tumor cells in the lymph node by detecting an image filling defect of at least a portion of the lymph node.

27. The method of claim 23, wherein the dendrimer conjugate is DAB-G5, PAMAM-G4, or PAMAM-G6.

28. The method of claim 27, wherein the dendrimer conjugate is PAMAM-G6.

29. The method of claim 23, wherein the tumor is a breast tumor.

30. The method of claim 23, wherein the dendrimer conjugate further comprises an optical or fluorescent moiety and the path of lymph flow from the tumor is visually detected during surgical removal of a portion of the lymphatic system draining the tumor.

31. The method of claim 23, wherein administering an image-enhancing amount of the dendrimer conjugate comprises administering a dose of between about 0.0001 mmol Gd/kg of the subject's body weight and 1.0 mmol Gd/kg of the subject's body weight.

32. The method of claim 23, further comprising using the image of the lymphatic system draining the tumor to guide a needle to the location of the lymph node.

33. The method of claim 32, further comprising obtaining a sample of the lymph node with the needle and analyzing the sample for the presence of metastatic cancer cells.
